(19) United States
(12) Patent Application Publication
(10) Pub. No.: US 2011/0052700 A1
(43) Pub. Date: Mar. 3, 2011

(54) GASTRIC RETENTIVE PHARMACEUTICAL COMPOSITIONS FOR IMMEDIATE AND EXTENDED RELEASE OF LEVOSULPIRIDE

(75) Inventors: Chien-Hsuan Han, Sunnyvale, CA (US); Sui Yuen Eddie Hou, Foster City, CA (US); Chunhong Gu, Cupertino, CA (US)

(73) Assignee: DEPOMED, INC., Menlo Park, CA (US)

(21) Appl. No.: 12/872,529

(22) Filed: Aug. 31, 2010

Related U.S. Application Data
(60) Provisional application No. 61/238,346, filed on Aug. 31, 2009.
(60) Provisional application No. 61/363,174, filed on Jul. 9, 2010.

(51) Int. Cl. A61K 9/14; A61K 31/40; A61P 1/00

(52) U.S. Cl. 424/486; 514/428

(57) ABSTRACT

Gastric retentive oral dosage forms which provide both immediate and extended release of levosulpiride are described which may allow once- or twice-daily dosing. Methods of treatment using the dosage forms and methods of making the dosage forms are also described.

This patent application claims the benefit of priority to U.S. Provisional Patent Application No. 61/238,346 filed on Aug. 31, 2009 and U.S. Provisional Patent Application No. 61/363,174 filed on Jul. 9, 2010, both of which are incorporated in their entirety herein by reference.

### TECHNICAL FIELD

The present subject matter relates generally to dosage forms for both immediate release and extended release of levosulpiride into the stomach of a patient in the fed mode and to methods of treatment using the dosage forms.

### BACKGROUND

Levosulpiride, a substituted benzamide, is the levorotatory (S) enantiomer of sulpiride. Sulpiride compounds have the general formula:

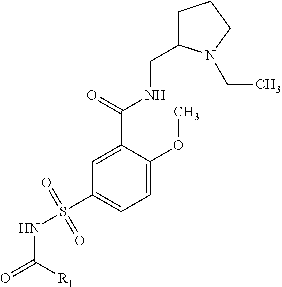

Levosulpiride is a selective dopamine D₂-receptor antagonist with prokinetic activity and has proven effective for the treatment of functional dyspepsia. Dyspepsia has been recently considered as a biopsychosocial disorder with the dysregulation of the brain-gut axis being central in the origin of the disease. The pathophysiology of functional dyspepsia is unknown, but a number of mechanisms have been suggested. There is considerable evidence to suggest an association between disordered motility and symptom production in functional dyspepsia. Motor dysfunction includes antral hypomotility and delayed gastric emptying, myoelectrical abnormalities of the gastric rhythm, abnormal tone (impaired gastric accommodation), or maldistribution of food within the stomach. Although the abnormal gastrointestinal motility may be the cause of the symptoms, the focus of research is shifting more toward sensory dysfunction as a primary abnormality, particularly selective visceral hypersensitivity to mechanical distension, acid hypersensitivity, or abnormal central processing of nociceptive stimuli. According to motor and/or sensory functional abnormalities causing dyspeptic symptoms, treatment options with prokinetics, serotonergic agents, antacids, and pain modulating medications have been proposed, although proton-pump inhibitor drugs (PPIs), histamine-2 receptor antagonists, and prokinetic agents are the most commonly used.

Levosulpiride and sulpiride, which are prokinetic agents, have been approved for use in several countries for the treatment of dyspepsia, chemotherapy-induced nausea and vomiting, and for psychotic disorders such as schizophrenia. However, at this time, neither levosulpiride nor sulpiride has been approved for therapeutic use in the United States despite their lack of side effects and their clinical efficacy.

Sulpiride is practically insoluble in water resulting in low absorption and bioavailability for the oral pharmaceutical formulations, and large injection volumes for the parenteral formulations. This, in turn, results in injectable products that have the potential to cause discomfort after injection to a patient.

Thus it would be of great use to manufacture an oral dosage form which is able to provide prolonged and steady levels of levosulpiride. Particularly advantageous would be a gastric retentive oral dosage form which provide extended release of an active agent upstream of absorption sites in the small intestine. Such dosage forms have been previously described, for example, in Gusler et al. (U.S. Pat. No. 6,723,34), Berner et al. (U.S. Pat. No. 6,488,962), Shell et al., (U.S. Pat. No. 6,340,475) and Shell et al. (U.S. Pat. No. 6,635,280). These formulations make use of one or more hydrophilic polymers which swell upon intake of water from gastric fluid. Thus, when administered in the fed mode, when the size of pyloric sphincter is reduced, the dosage form will swell to a size to be retained in the stomach for a minimum of four hours or more. These formulations may be designed to produce desired release and delivery profiles for both highly soluble and poorly soluble drugs.

As presently disclosed, gastric retentive dosage forms are formulated to provide both an immediate and an extended release of levosulpiride to allow prolonged delivery of therapeutically effective amounts of levosulpiride.

### BRIEF SUMMARY

The present disclosure provides, among other aspects, gastric retentive dosage forms for oral administration to a subject, such as a human patient, for the treatment of gastrointestinal disorders including, but not limited to, functional dyspepsia and gastroesophageal reflux disease (GERD). The dosage form in some embodiments is a gastric retentive dosage form that contains a first dose of levosulpiride as an extended release (“ER”) layer and optionally contains a second dose of levosulpiride as an immediate release (“IR”) layer. In other embodiments, the gastric retentive dosage form further provides a delayed burst release of levosulpiride.

In one aspect, the ER layer of the dosage form comprises the first dose of levosulpiride dispersed in a polymer matrix comprising at least one hydrophilic polymer. Upon administration, the polymer matrix swells upon imbibition of fluid to a size sufficient such that the ER portion of the dosage form is retained in a stomach of a subject in a fed mode and the first dose of levosulpiride is released over an extended period of time.

In one aspect, a bilayer tablet comprising an ER layer and an IR layer is provided. In one embodiment, the bilayer tablet comprises a total of about 20 mg (milligrams) to 50 mg levosulpiride. In another embodiment, the bilayer tablet comprises a total of about 30 mg, 35 mg, 40 mg, 45 mg, or 50 mg levosulpiride. In yet another embodiment, the bilayer tablet comprises a total of about 37.5 mg levosulpiride. In still another embodiment, the bilayer tablet comprises an ER layer comprising about 25 mg to 40 mg, 30 mg to 38 mg, 35 mg to 38 mg, 25 mg, 26 mg, 27 mg, 28 mg, 29 mg, 30 mg, 31 mg, 32 mg, 33 mg, 34 mg, 35 mg, 36 mg, or 37 mg levosulpiride, and an IR layer comprising about 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg, or 10.0 mg levosulpiride.

In one embodiment, the ER layer comprises about 0.1 wt % (weight %), 0.2 wt %, 0.3 wt %, 0.5 wt %, 0.8 wt %, or 1.0 wt % binder. In yet another embodiment, the ER layer comprises about 0.1 wt % to about 2.0 wt % or about 0.1 wt % to about 1.0 wt % of a binder.

In one embodiment, the ER layer comprises about 480 mg, 500 mg, 520 mg, 540 mg, 560 mg, 580 mg, 600 mg, or 620 mg of one or more hydrophilic polymers. In another embodiment, the ER layer comprises about 40 wt % to about 90 wt % or about 50 wt % to about 80 wt % of a hydrophilic polymer. In yet another embodiment, the ER layer comprises about 40 wt/%, 45 wt %, 50 wt %, 55 wt %, 60 wt %, 65 wt %, 70 wt %, 75 wt %, 80 wt %, 85 wt %, or 90 wt % of a hydrophilic polymer.

In one embodiment, the ER layer comprises about 0.5 wt % to about 2.5 wt % of a lubricant. In yet another embodiment, the ER layer comprises about 0.5 wt %, 1.0 wt %, 1.5 wt %, 2.0 wt %, or 2.5 wt % of a lubricant.

In one embodiment, the IR layer comprises about 1.0 wt %, 2.0 wt %, 3.0 wt %, 4.0 wt %, 5.0 wt %, 6.0 wt %, 7.0 wt %, 8.0 wt %, 9.0 wt %, or 10.0 wt % of a disintegrant. In another embodiment, the IR layer comprises about 1.0 wt % to 10.0 wt % or about 1.0 wt % to 5.0 wt % of a disintegrant.

In another embodiment, the IR layer comprises at least one binder. In another embodiment, the IR layer comprises about 2 wt %, 3 wt %, 4 wt %, 5 wt %, 6 wt %, 7 wt %, 8 wt %, 9 wt % or 10 wt % of a binder. In yet another embodiment, the IR layer comprises about 4 wt % to 10 wt % of a binder. In still another embodiment, the IR layer comprises about 5 wt % to 8 wt % of a binder.

In one embodiment, the bilayer tablet comprises a total of about 50 mg to 100 mg or about 60 to about 80 mg levosulpiride. In another embodiment, the bilayer tablet comprises a total of about 50 mg, 60 mg, 70 mg, 75 mg, 80 mg, 90 mg or 100 mg levosulpiride. In yet another embodiment, the bilayer tablet comprises a total of about 50 mg, 55 mg, 60 mg, In still another embodiment, the bilayer tablet comprises an ER layer comprising about 30 mg, 35 mg, 40 mg, 45 mg, 50 mg, 55 mg, 60 mg, or 65 mg levosulpiride. In yet another embodiment, the bilayer tablet comprises an IR layer comprising about 15 mg, 20 mg, 25 mg, 30 mg, 35 mg, 40 mg, 45 mg, or 50 mg levosulpiride.

In one embodiment, the ER layer comprises about 3.0 wt %, 3.2 wt %, 3.4 wt %, 3.6 wt %, 3.8 wt %, 4.0 wt %, 4.2 wt %, 4.4 wt %, 4.6 wt %, 4.8 wt %, 5.0 wt %, 5.2 wt %, 5.4 wt %, 5.6 wt %, 5.8 wt %, 6.0 wt %, 6.2 wt %, 6.4 wt %, 6.6 wt %, 6.8 wt %, 7.0 wt %, 7.2 wt %, 7.4 wt %, 7.6 wt %, 7.8 wt %, or 8.0 wt % of levosulpiride. In another embodiment, the IR layer comprises about 1.5 wt %, 2.0 wt %, 2.5 wt %, 3.0 wt %, 3.5 wt %, 4.0 wt %, 4.5 wt %, 5.0 wt %, 5.5 wt %, 6.0 wt %, 6.5 wt %, 7.0 wt %, 7.5 wt %, 8.0 wt %, 8.5 wt %, 9.0 wt %, 9.5 wt %, 10.0 wt %, 10.5 wt %, 11.0 wt %, 11.5 wt %, or 12.0 weight % of levosulpiride.

In one embodiment, the ER and/or IR layer comprises a chelating agent. Examples of chelating agents include ethylenediamine tetracetic acid (EDTA) and its salts (including a sodium salt), N-(hydroxy-ethyl)ethylenediaminetriacetic acid, nitrilotriacetic acid (NIA), ethylene-bis(oxyethylene-nitrilo)tetraacetic acid, 1,4,7,10-tetraazacyclodo-decane-N,N′,N″,N′″-tetraacetic acid, 1,4,7,10-tetraaza-cyclododecane-N,N′,N″-triacetic acid, 1,4,7-tris(carboxymethyl)-10-(2′-hydroxypropyl)-1,4,7,10-tetraazocyclodecane, 1,4,7-triazacyclonane-N,N′,N″-triacetic acid, 1,4,8,11-tetraazacyclotetra-decane-N,N′,N″,N′″-tetraacetic acid; diethylenetriamine-pentaacetic acid (DTPA), ethylenedicysteine, bis(aminoethanethiol)carboxylic acid, triethylenetetraamine-hexaacetic acid, and 1,2-diaminocyclohexane-N,N,N′,N′-tetraacetic acid. The chelating agent may be present in the dosage form in an amount that is about 0.01 wt % to about 0.10 wt % or about 0.02 to about 0.08 wt % of the tablet. Alternatively, the table may comprise about 0.01 wt %, 0.02 wt %, 0.03 wt %, 0.04 wt %, 0.05 wt %, 0.06 wt %, 0.07 wt %, 0.08 wt %, 0.09 wt % or 0.10 wt % of the chelating agent.

In one embodiment, the ER and/or IR layer of the dosage form comprises an anti-oxidant which is ascorbic acid, citric acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, or propylgallate. In another embodiment, the antioxidant is present in the ER and/or IR portion of the dosage form at a wt % ranging from about 0.10 wt % to about 0.20 wt %, or from about 0.05 wt % to about 0.30 wt %. In yet another embodiment, the antioxidant is present in the ER and/or IR portion of the dosage form at a wt % of about 0.01 wt %, 0.05 wt %, 0.10 wt %, 0.15 wt %, 0.20 wt %, 0.25 wt %, 0.35 wt %, 0.50 wt %, 0.75 wt %, 1.00 wt %, 2.00 wt %, 3.00 wt % or 4.00 wt % of the ER portion.

In one embodiment, a tablet comprising an ER layer is provided. In another embodiment, the tablet does not comprise an IR layer.

In one embodiment, the ER layer comprises a hydrophilic polymer having an average molecular weight ranging from about 200,000 Da (Daltons) to about 7,000,000 Da, about 900,000 Da to about 5,000,000 Da, about 2,000,000 Da to about 5,000,000 Da, from about 4,000,000 Da to about 5,000,000 Da, from about 2,000,000 Da to about 4,000,000 Da, from about 900,000 Da to about 5,000,000 Da, or from about 900,000 Da to about 4,000,000 Da. In another embodiment, the ER layer comprises a hydrophilic polymer having an average molecular weight of about 200,000 Da, 600,000 Da, 900,000 Da, 1,000,000 Da, 2,000,000 Da, 4,000,000 Da, 5,000,000 Da, 7,000,000 Da or 10,000,000 Da.

In one embodiment, the ER layer comprises a hydrophilic polymer having an average viscosity ranging from about 4,000 cp (centipoise) to about 200,000 cp, from about 50,000 cp to about 200,000 cp, or from about 80,000 cp to about 120,000 cp as measured as a 2% weight per volume in water at 20° C.

In one embodiment, the ER layer comprises a total amount of hydrophilic polymer which is between about 100 mg and 225 mg or about 125 mg to about 200 mg. In another embodiment, the total amount of hydrophilic polymer in the ER layer is about 100 mg, 110 mg, 115 mg, 120 mg, 125 mg, 130 mg, 135 mg, 140 mg, 145 mg, 150 mg, 155 mg, 160 mg, 165 mg, 170 mg, 175 mg or 180 mg. In yet another embodiment, the total amount of hydrophilic polymer in the ER layer is present in an amount which is about 50 wt % to about 40 wt % or about 10 wt % to about 30 wt % of the ER layer. In yet another embodiment, the total amount of hydrophilic polymer in the ER layer is present in an amount which is about 10 wt %, 12 wt %, 14 wt %, 15 wt %, 17 wt %, 18 wt %, 20 wt %, 22 wt %, 24 wt %, 25 wt %, 27 wt % or 30 wt % of the ER layer.

In one embodiment, the at least one hydrophilic polymer in the ER layer is a polyalkylene oxide. In another embodiment, the hydrophilic polymer is poly(ethylene oxide). In yet another embodiment, the at least one hydrophilic polymer in the ER layer is a cellulose. In yet another embodiment, the cellulose is hydroxypropyl methylcellulose. In yet another embodiment, the ER layer comprises two hydrophilic polymers in a ratio of 3:1, 3:1.5, 3:2, 2:1, 2:1.5, 1:1, 1:1.5, 1:2, 1:2.5, or 1:3.

In one embodiment, the levosulpiride is released from the ER layer over a time period of about 6 to 12 h (hours) in vitro. about 6 to 11 h, about 7 to 10 h, about 8 to 9 h, about 8 to 10 h, or about 9 to 10 h in vitro.

In one embodiment, the levosulpiride is released from the ER layer over a time period of about 6 to 12 h in vivo. about 6 to 11 h, about 7 to 10 h, about 8 to 9 h, about 8 to 10 h, or about 9 to 10 h. In another embodiment, levosulpiride is delivered to the small intestine of the subject over a time period of at least about 4 h, 5 h, 6 h, 7 h, 8 h, 9 h, 10 h, 11 h, 12 h, or 13 h.

In one embodiment, the levosulpiride is released from the ER layer via diffusion. In another embodiment, the levosulpiride is released from the ER layer via erosion. In yet another embodiment, the levosulpiride is released from the ER layer via a combination of diffusion and erosion.

In one embodiment, the ER layer comprises a binder which is polyvinylpyrrolidone, polyvinylalcohol, ethyl cellulose, or polyethylene glycol, hydroxyalkyl cellulose, or hydroxypropyl alkyl cellulose. In yet another embodiment, the polyvinylpyrrolidone is povidone, copovidone, or crospovidone. In yet another embodiment, the ER layer comprises a combination of more than one binder.

In one embodiment, the ER layer further comprises a lubricant which is magnesium stearate, calcium stearate, sodium stearyl fumarate, stearic acid, stearyl behenate, glyceryl behenate, or polyethylene glycol.

In one embodiment, the ER layer comprises a lubricant which is present in an amount ranging from about 0.3 to 10 mg. In yet another embodiment, the amount of lubricant in the ER layer is about 0.3 mg, 0.4 mg, 0.5 mg, 0.6 mg, 0.7 mg, 0.8 mg, 0.9 mg, 1.0 mg, 1.2 mg, 1.4 mg, 1.6 mg, 1.8 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 or 10 mg. In yet another embodiment, the amount of lubricant in the ER layer is about 0.4 wt %, 0.5 wt %, 0.6 wt %, 0.7 wt %, 0.8 wt %, 0.9 wt %, 1.0 wt %, 1.2 wt %, 1.4 wt %, 1.6 wt %, 1.8 wt %, 2.0 wt %, 2.2 wt %, 2.4 wt %, or 2.5 wt % of the ER layer.

In one embodiment, the ER layer comprises one or more additional excipients which are diluents, coloring agents, flavoring agents, and/or glidants.

In one embodiment, the dosage form is a single layer tablet comprising an ER layer.

In another embodiment, the ER layer comprises about 35.0 mg, 37.5 mg, 40.0 mg, 45.0 mg, 50.0 mg, 55.0 mg, 60.0 mg, 65.0 mg, 70.0 mg, 75.0 mg, 80.0 mg, 85.0 mg, 90.0 mg or 95.0 mg levosulpiride. In another embodiment, the ER layer is coated by an IR layer containing levosulpiride.

In some embodiments, the single, bilayer, multilayer or coated tablet has a friability of no greater than about 0.1%, 0.2% 0.3%, 0.4%, 0.5%, 0.7% or 1.0%.

In one aspect, the dosage form comprising a drug optionally comprises an outer IR layer comprising a first dose of the drug, comprises an intermediate ER layer comprising a second dose of the drug and comprises an inner IR layer comprising a third dose of the drug. The third dose of drug is released as a “pulse” or “burst.” In another embodiment the drug is levosulpiride. In still another embodiment, the drug in the outer, intermediate and inner layers is the same or is different in two or three of the layers.

In one embodiment, the optional outer IR layer releases essentially all, or at least 90% of the first dose of the drug within 15 minutes of oral administration of the dosage form. In another embodiment, the intermediate ER layer releases the second dose of the drug over a time period of about 2 h to 10 h, about 3 h to 8 h, about 4 h to 7 h, about 5 h to 9 h, about 5 h to 8 h, or about 4 h to 6 h. In yet another embodiment, the intermediate ER layer releases the second dose of the drug over a time period of about 3 h, 4 h, 5 h, 6 h, 7 h, 8 h, 9 h or 10 h. In still another embodiment, the inner IR layer releases the third dose of the drug over a time period of about 1 min to 90 min, about 5 min to 60 min, about 10 min to 45 min, or about 20 min to 40 min.

In one embodiment, the intermediate ER layer comprises a swellable hydrophilic polymer. In another embodiment, the swellable hydrophilic polymer has an average molecular weight ranging from about 900,000 Da to 10 million Da, about 1 million Da to 7 million Da, about 2 million Da to 5 million Da, about 1 million Da to 2 million Da, about 1 million Da to 4 million Da or about 2 million Da to 4 million Da.

In one embodiment, the swellable hydrophilic polymer is present in the intermediate ER layer at a wt % of about 20 wt % to 50 wt %, about 20 wt % to 40 wt %, or about 30 wt % to 40 wt %. In another embodiment, the swellable hydrophilic polymer is present in the intermediate ER layer at a wt % of about 20 wt %, about 25 wt %, about 30 wt %, about 35 wt %, about 40 wt %, about 45 wt % or about 50 wt %.

In one embodiment, the wt % of the drug present in the intermediate ER layer comprises about 1 wt % to 5 wt %, about 2 wt % to 4 wt %, or about 2 wt % to 3 wt % of the second dose of the drug. In another embodiment, the wt % of the drug present in the intermediate ER layer comprises about 2.0 wt %, 2.5 wt %, 3.0 wt %, 3.5 wt %, 4.0 wt %, 4.5 wt % or 5.0 wt % of the second dose of the drug.

In one embodiment, the intermediate ER layer further comprises one or more excipients selected from the group consisting of at least one binder, at least one lubricant, at least one filler, at least one metal ion chelator, and at least one antioxidant.

In one embodiment, the intermediate ER layer swells in fluid. In another embodiment, the dosage form swells upon imbibition of the fluid to a size sufficient for gastric retention in the subject in a fed mode. In another embodiment, the size sufficient for gastric retention in the subject in a fed mode is defined by a smallest diameter of the dosage form of at least about 1 cm, about 1.2 cm, about 1.5 cm, about 1.8 cm, about 2.0 cm, about 2.5 cm, about 3.0 cm, about 3.5 cm, about 4.0 cm, about 5.0 cm or greater.

In one embodiment, after oral administration to a subject, the intermediate ER layer swells upon imbibition of fluid to a size which is at least 120%, 130%, 140%, 150%, 175% or 200% greater than the size of the intermediate ER layer prior to imbibition of fluid within 15 minutes of the imbibition.

In one embodiment, the intermediate ER layer of the oral dosage form prior to oral administration completely encases or surrounds the inner IR layer.

In one embodiment, the wt % of the drug present in the inner IR layer comprises about 90 wt %, about 95 wt %, about 96 wt %, about 97 wt %, about 98 wt %, or about 99 wt % of the third dose of the drug.

In one embodiment, the inner IR layer further comprises one or more excipients selected from the group consisting of at least one binder, at least one antioxidant, and at least one disintegrant.

In one aspect, the oral dosage form possesses a stability that meets the requirements of the FDA. In some embodiments, the oral dosage form has a hardness of at least about 10 kiloponds (kp). In some embodiments, the tablet has a hardness of about 9 kp to about 25 kp, or about 12 kp to about 20 kp. In further embodiments, the tablet has a hardness of about 11 kp, 12 kp, 13 kp, 14 kp, 15 kp, 16 kp, 17 kp, 18 kp, 19 kp, 20 kp, 21 kp, 22 kp, 23 kp, 24 kp or 25 kp. In some embodiments, the tablets have a content uniformity of from about 85 to about 115 percent by weight or from about 90 to about 110 percent by weight, or from about 95 to about 105 percent by weight. In other embodiments, the content uniformity has a relative standard deviation (RSD) equal to or less than about 3.5%, 3.0%, 2.5%, 2.0%, 1.5%, 1.0% or 0.5%.

In one embodiment, about 90% to about 100% of the first dose of levosulpiride is released within 10 minutes, 15 minutes, 30 minutes, 45 minutes or 60 minutes after oral administration.

In one embodiment, the ER layer swells upon imbibition of fluid from gastric fluid to a size which is at least about 15%, 20%, 25%, 30%, 35% 40%, 45%, 50%, 65%, 75%, 85% or 100% larger than the size of the ER layer prior to imbibition of fluid within 45-60 minutes after in contact with the fluid.

In one embodiment, the dosage form provides a dissolution profile wherein between about 50% to about 85%, about 55% to about 80% or about 35% to about 55% of the second dose of levosulpiride remains in the ER layer between about 1 and 2 hours after administration. In one embodiment, not more than about 15%, 20%, 30%, or 40% of the second dose of levosulpiride is released within about the first hour. In another embodiment, not more than about 25%, 30%, 35%, 40%, 45%, 50%, 55%, 60%, 65%, 70%, 75%, or 80% of the second dose of levosulpiride is released within about 4 hours. In yet another embodiment, not less than about 50%, 55%, 60%, 65%, 70%, or 75% is released after about 6 hours. In yet another embodiment, not less than about 60% is released after about 6 hours.

In one aspect, a method of making a gastric retentive dosage form comprising levosulpiride and at least one hydrophilic polymer is provided.

In one embodiment, a method of making the IR layer of the dosage form comprising levosulpiride powder with one or more binders and one or more disintegrants is provided. In another embodiment, the method of making the ER layer comprising the levosulpiride powder with a solution of binder is provided. In yet another embodiment, the method of making the IR and ER layers further comprises granulating the IR and ER mixtures produced by the granulation processes, then screening the granules. In yet another embodiment, the method of making the IR and ER layers further comprises blending the granules with additional excipients.

In one aspect, a method for treating dyspepsia or gastroesophageal reflux disease is provided.

In one embodiment, a gastric retained dosage form comprising levosulpiride and at least one swellable polymer is administered to a subject suffering from or diagnosed with dyspepsia or gastroesophageal reflux disease. In another embodiment, the gastric retained dosage form comprising levosulpiride is administered to a subject suffering from schizophrenia.

In one embodiment, a gastric retained dosage form is administered to a subject in a fed mode. In another embodiment, the dosage form is administered with a meal to a subject once in a 24 hour period. In other embodiments, the dosage form is administered with a meal to the subject twice or thrice in a 24 hour period. In yet another embodiment, the dosage form is administered with a meal to a subject once or twice in a 24 hour period for 2, 3, 4, 5, 6, 7, 8 or more days.

### BRIEF DESCRIPTION OF THE DRAWINGS

FIG. 1 is a graph showing dissolution release profiles for tablets containing 75 mg levosulpiride.

FIG. 2 is a graph showing dissolution release profiles for tablets containing 37.5 mg levosulpiride.

FIG. 3 is a graph showing dissolution release profiles for tablets having both IR and ER layers containing 6.5 and 31 mg levosulpiride, respectively.

FIG. 4. shows results of in vitro dissolution rate studies for bilayer tablets having 75 mg levosulpiride, at pH 1.2 (▪), 4.5 (♦) and 6.8 (▴).

FIG. 5 shows results of in vitro dissolution rate studies for bilayer tablets having 37.5 mg levosulpiride, at pH 1.2 (▪), 4.5 (♦) and 6.8 (▴).

FIG. 6 is a plot of a linear regression of percentage drug release (ER component) as a function of time for in vitro dissolution rate studies for bilayer tablets having 75 mg levosulpiride, at pH 1.2 (▪), 4.5-(♦) and 6.8 (▴).

FIG. 7 is a plot of a linear regression of percentage drug release (ER component) as a function of time for in vitro dissolution rate studies for bilayer tablets having 37.5 mg levosulpiride, at pH 1.2 (▪), 4.5 (♦) and 6.8 (▴).

FIG. 8 shows pharmacokinetic simulation results comparing q.d. administration of the gastric retained bilayer tablet containing 75 mg levosulpiride (solid line) with t.i.d. administration of the immediate release commercial product (dashed line).

FIG. 9 shows pharmacokinetic simulation results comparing b.i.d. administration of the gastric retained bilayer tablet containing 37.5 mg levosulpiride (solid line) with t.i.d. administration of the immediate release commercial product (dashed line).

FIGS. 10A-D show HPLC profiles resulting from impurity analysis.

FIG. 11 illustrates a mechanism whereby a dosage form swells and releases drug from an ER layer or shell and from an IR core.

FIG. 12 shows results of in vitro dissolution rate studies for ER shell/IR core tablets having 20 wt % PEO N-1105 (▪), 40 wt % PEO N-1105 (▴), or 40 wt % PEO N-60K (♦).

### DETAILED DESCRIPTION

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art.

### I. DEFINITIONS

It must be noted that, as used in this specification, the singular forms “a,” “an,” and “the” include plural referents unless the context clearly dictates otherwise.

Compounds useful in the compositions and methods include those described herein in any of their pharmaceutically acceptable forms, including isomers such as diastereomers and enantiomers, salts, solvates, and polymorphs, as well as racemic mixtures and pure isomers of the compounds described herein, where applicable.

“Optional” or “optionally,” as used herein, means that the subsequently described element, component or circumstance may or may not occur, so that the description includes instances where the element, component, or circumstance occurs and instances where it does not.

The terms “subject,” “individual” or “patient” are used interchangeably herein and refer to a vertebrate, preferably a mammal. Mammals include, but are not limited to, humans.

The term “about”, particularly in reference to a given quantity, is meant to encompass deviations of plus or minus five percent.

The term “fed mode,” as used herein, refers to a state which is typically induced in a patient by the presence of food in the stomach, the food-giving rise to two signals, one that is said to stem from stomach distension and the other a chemical signal based on food in the stomach. It has been determined that once the fed mode has been induced, larger particles are retained in the stomach for a longer period of time than smaller particles; thus, the fed mode is typically induced in a patient by the presence of food in the stomach. The fed mode is initiated by nutritive materials entering the stomach upon the ingestion of food. Initiation is accompanied by a rapid and profound change in the motor pattern of the upper GI tract, over a period of 30 seconds to one minute. The change is observed almost simultaneously at all sites along the G.I. tract and occurs before the stomach contents have reached the distal small intestine. Once the fed mode is established, the stomach generates 3-4 continuous and regular contractions per minute, similar to those of the fasting mode but with about half the amplitude. The pylorus is partially open, causing a sieving effect in which liquids and small particles flow continuously from the stomach into the intestine while indigestible particles greater in size than the pyloric opening are retropelled and retained in the stomach. This sieving effect thus causes the stomach to retain particles exceeding about 1 cm in size for about 4 to 6 hours. Administration of a dosage form “with a meal,” as used herein, refers to administration before, during or after a meal, and more particularly refers to administration of a dosage form about 1, 2, 3, 4, 5, 10, 15 minutes before commencement of a meal, during the meal, or about 1, 2, 3, 4, 5, 10, 15 minutes after completion of a meal.

A drug “release rate,” as used herein, refers to the quantity of drug released from a dosage form or pharmaceutical composition per unit time, e.g., milligrams of drug released per hour (mg/hr). Drug release rates for drug dosage forms are typically measured as an in vitro rate of dissolution, i.e., a quantity of drug released from the dosage form or pharmaceutical composition per unit time measured under appropriate conditions and in a suitable fluid. The specific results of dissolution tests claimed herein are performed on dosage forms or pharmaceutical compositions in a USP Type II apparatus and immersed in 900 ml of simulated intestinal fluid (SIF) at pH 6.8 and equilibrated in a constant temperature water bath at 37° C. Suitable aliquots of the release rate solutions are tested to determine the amount of drug released from the dosage form or pharmaceutical composition. For example, the drug can be assayed or injected into a chromatographic system to quantify the amounts of drug released during the testing intervals.

The terms “hydrophilic” and “hydrophobic” are generally defined in terms of a partition coefficient P, which is the ratio of the equilibrium concentration of a compound in an organic phase to that in an aqueous phase. A hydrophilic compound has a P value less than 1.0, typically less than about 0.5, where P is the partition coefficient of the compound between octanol and water, while hydrophobic compounds will generally have a P greater than about 1.0, typically greater than about 5.0. The polymeric carriers herein are hydrophilic, and thus compatible with aqueous fluids such as those present in the human body.

The term “polymer” as used herein refers to a molecule containing a plurality of covalently attached monomer units, and includes branched, dendrimeric, and star polymers as well as linear polymers. The term also includes both homopolymers and copolymers, e.g., random copolymers, block copolymers and graft copolymers, as well as uncrosslinked polymers and slightly to moderately to substantially crosslinked polymers, as well as two or more interpenetrating cross-linked networks.

The term “swellable polymer,” as used herein, refers to a polymer that will swell in the presence of a fluid. It is understood that a given polymer may or may not swell when present in a defined drug formulation. Accordingly, the term “swellable polymer” defines a structural feature of a polymer which is dependent upon the composition in which the polymer is formulated. Whether or not a polymer swells in the presence of fluid will depend upon a variety of factors, including the specific type of polymer and the percentage of that polymer in a particular formulation. For example, the term “polyethylene oxide” or “PEO” refers to a polyethylene oxide polymer that has a wide range of molecular weights. PEO is a linear polymer of unsubstituted ethylene oxide and has a wide range of viscosity-average molecular weights. Examples of commercially available PEOs and their approximate molecular weights are: POLYOX® NF, grade WSR coagulant, approximate molecular weight 5 million, POLYOX® grade WSR 301, approximate molecular weight 4 million, POLYOX® grade WSR 303, approximate molecular weight 7 million, POLYOX® grade WSR N-60K, approximate molecular weight 2 million, and POLYOX® grade N-80K, approximate molecular weight 200,000. It will be understood by a person with ordinary skill in the art that an oral dosage form which comprises a swellable polymer will swell upon imbibition of water or fluid from gastric fluid.

The terms “swellable” and “bioerodible” (or simply “erodible”) are used to refer to the polymers used in the present dosage forms, with “swellable” polymers being those that are capable of absorbing water and physically swelling as a result, with the extent to which a polymer can swell being determined by the molecular weight or degree of crosslinking (for crosslinked polymers), and “bioerodible” or “erodible” polymers referring to polymers that slowly dissolve and/or gradually hydrolyze in an aqueous fluid, and/or that physically disentangle or undergo chemical degradation of the chains themselves, as a result of movement within the stomach or GI tract.

The term “friability,” as used herein, refers to the ease with which a tablet will break or fracture. The test for friability is a standard test known to one skilled in the art. Friability is measured under standardized conditions by weighing out a certain number of tablets (based on USP requirement), placing them in a rotating Plexiglas drum in which they are lifted during replicate revolutions by a radial lever, and then dropped about 8 inches. After replicate revolutions (typically 100 revolutions based on USP requirement), the tablets are reweighed and the percentage of formulation abraded or chipped is calculated. The friability of the tablets, of the present invention, is preferably in the range of about 0% to 3%, and values about 1%, or less, are considered acceptable for most drug and food tablet contexts. Friability which approaches 0% is particularly preferred.

The term “tap density” or “tapped density,” as used herein, refers to a measure of the density of particles used to make the dosage form, wherein the particles may be, for example, powders or granules. The tapped density of a pharmaceutical particles is determined using a tapped density tester, which is set to tap the powder particles at a fixed impact force and frequency. Tapped density by the USP method is determined by a linear progression of the number of taps.

The term “bulk density,” as used herein, refers to a property of particles and is defined as the mass of many particles of the material divided by the total volume they occupy. The total volume includes particle volume, inter-particle void volume and internal pore volume.

The term “capping,” as used herein, refers to the partial or complete separation of top or bottom crowns of the tablet main body. For multilayer tablets, capping refers to separation of a portion of an individual layer within the multilayer tablet. Unintended separation of layers within a multilayer tablet prior to administration is referred to herein as “splitting.”

The term “content uniformity,” as used herein refers to the testing of compressed tablets to provide an assessment of how uniformly the micronized or submicron active ingredient is dispersed in the powder mixture. Content uniformity is measured by use of USP Method (General Chapters, Uniformity of Dosage Forms), unless otherwise indicated. A plurality refers to five, ten or more tablet compositions.

The terms “effective amount” or a “therapeutically effective amount” refer to the amount of drug or pharmacologically active agent to provide the desired effect without toxic effects. The amount of an agent that is “effective” may vary from individual to individual, depending on the age, weight, general condition, and other factors of the individual. An appropriate “effective” amount in any individual may be determined by one of ordinary skill in the art using routine experimentation. An “effective amount” of an agent can refer to an amount that is either therapeutically effective or prophylactically effective or both.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. The term “pharmacologically active” (or simply “active”) as in a “pharmacologically active” derivative, refers to a derivative having the same type of pharmacological activity as the parent compound and/or drug and about equivalent in degree. When the term “pharmaceutically acceptable” is used to refer to a derivative (e.g., a salt) of an active agent, it is to be understood that the compound is pharmacologically active as well. When the term, “pharmaceutically acceptable” is used to refer to an excipient, it implies that the excipient has met the required standards of toxicological and manufacturing testing or that it is on the Inactive Ingredient Guide prepared by the FDA, or comparable agency.

The terms “drug,” “active agent,” “therapeutic agent,” and/or “pharmacologically active agent” are used interchangeably herein to refer to any chemical compound, complex or composition that is suitable for oral administration and that has a beneficial biological effect, preferably a therapeutic effect in the treatment or prevention of a disease or abnormal physiological condition. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent,” and “drug” are used, then, or when a particular active agent is specifically identified, it is to be understood that applicants intend to include the active agent per se as well as pharmaceutically acceptable, pharmacologically active salts, esters, amides, prodrugs, metabolites, analogs, etc.

The term “dosage form” refers to the physical formulation of the drug for administration to the patient. Dosage forms include without limitation, tablets, capsules, caplets, liquids, syrups, lotions, lozenges, aerosols, patches, enemas, oils, ointments, pastes, powders for reconstitution, sachets, solutions, sponges, and wipes. Within the context of the present invention, a dosage form comprising a levosulpiride formulation will generally be administered to patients in the form of tablets.

The term “dosage unit” refers to a single unit of the dosage form that is to be administered to the patient. The dosage unit will be typically formulated to include an amount of drug sufficient to achieve a therapeutic effect with a single administration of the dosage unit although where the size of the dosage form is at issue, more than one dosage unit may be necessary to achieve the desired therapeutic effect. For example, a single dosage unit of a drug is typically, one tablet, one capsule, or one tablespoon of liquid. More than one dosage unit may be necessary to administer sufficient drug to achieve a therapeutic effect where the amount of drug causes physical constraints on the size of the dosage form.

“Delayed release” dosage forms are a category of modified release dosage forms in which the release of the drug is delayed after oral administration for a finite period of time after which release of the drug is unhindered. Delayed release dosage forms are frequently used to protect an acid-labile drug from the low pH of the stomach or where appropriate to target the GI tract for local effect while minimizing systemic exposure. Enteric coating is frequently used to manufacture delayed release dosage forms.

The terms “sustained release,” and “extended release” are used interchangeably herein to refer to a dosage form that provides for gradual release of a drug over an extended period of time. With extended release dosage forms, the rate of release of the drug from the dosage form is reduced in order to maintain therapeutic activity of the drug for a longer period of time or to reduce any toxic effects associated with a particular dosing of the drug. Extended release dosage forms have the advantage of providing patients with a dosing regimen that allows for less frequent dosing, thus enhancing compliance. Extended release dosage forms can also reduce peak-related side effects associated with some drugs and can maintain therapeutic concentrations throughout the dosing period thus avoiding periods of insufficient therapeutic plasma concentrations between doses.

The term “modified release” refers to a dosage form that includes both delayed and extended release drug products. The manufacture of delayed, extended, and modified release dosage forms are known to ordinary skill in the art and include the formulation of the dosage forms with excipients or combinations of excipients necessary to produce the desired active agent release profile for the dosage form.

The “gastric retentive” oral dosage forms described herein are a type of extended release dosage form. Gastric retentive dosage forms are beneficial for the delivery of drugs with reduced absorption in the lower GI tract or for local treatment of diseases of the stomach or upper GI tract. For example, in certain embodiments of gastric retentive oral dosage forms of the present invention, the dosage form swells in the gastric cavity and is retained in the gastric cavity of a patient in the fed med so that the drug may be released for heightened therapeutic effect. See, Hou et al., Crit. Rev. Ther. Drug Carrier Syst. 20(6):459-497 (2003).

The in vivo “release rate” and in vivo “release profile” refer to the time it takes for the orally administered dosage form, or the active agent-containing layer of a bilayer or multilayer tablet (administered when the stomach is in the fed mode) or the content of the active ingredient to be reduced to 0-10%, preferably 0-5%, of its original size or level, as may be observed visually using NMR shift reagents or paramagnetic species, radio-opaque species or markers, or radiolabels, or determined mathematically, such as deconvolution, upon its plasma concentration profiles.

The term “AUC” (i.e., “area under the curve,” “area under the concentration curve,” or “area under the concentration-time curve”) is a pharmacokinetic term used to refer a method of measurement of bioavailability or extent of absorption of a drug based on a plot of an individual or pool of individual's blood plasma concentrations sampled at frequent intervals; the AUC is directly proportional to the total amount of unaltered drug in the patient's blood plasma. For example, a linear curve for a plot of the AUC versus dose (i.e., straight ascending line) indicates that the drug is being released slowly into the blood stream and is providing a steady amount of drug to the patient; if the AUC versus dose is a linear relationship this generally represents optimal delivery of the drug into the patient's blood stream. By contrast, a non-linear AUC versus dose curve indicates rapid release of drug such that some of the drug is not absorbed, or the drug is metabolized before entering the blood stream.

The term “Cmax” (i.e., “maximum concentration”) is a pharmacokinetic term used to indicate the peak concentration of a particular drug in the blood plasma of a patient.

The term “Tmax” (i.e., “time of maximum concentration” or “time of Cmax”) is a pharmacokinetic term used to indicate the time at which the Cmax is observed during the time course of a drug administration.

“Preventing,” in reference to a disorder or unwanted physiological event in a patient, refers specifically to inhibiting or significant reducing the occurrence of symptoms associated with the disorder and/or the underlying cause of the symptoms.

“Therapeutically effective amount,” in reference to a therapeutic agent, refers to an amount that is effective to achieve a desired therapeutic result. Therapeutically effective amounts of a given agent will typically vary with respect to factors such as the type and severity of the disorder or disease being treated and the age, gender, weight and other factors of the patient.

“Treating,” “treat,” and “treatment” refer to reduction in severity and/or frequency of symptoms, elimination of symptoms and/or underlying cause, prevention of the occurrence of symptoms and/or their underlying cause, and improvement or remediation of damage.

### II. GASTRIC RETENTIVE DOSAGE FORM FOR THE EXTENDED RELEASE OF LEVOSULPIRIDE

The pharmaceutical compositions described herein, i.e., gastric retained dosage forms comprising levosulpiride, provide both immediate release and extended or sustained release of levosulpiride to the upper gastrointestinal tract. The presently described dosage forms provide for extended release of levosulpiride in the stomach wherein the dosage forms are comprised of a polymer matrix that swells upon imbibition of fluid to a size sufficient for gastric retention. Thus, in formulating the dosage forms, it is desirable to provide the properties which simultaneously allow: a) an extent of swelling to provide gastric retention over an extended period, and b) a rate of swelling and erosion that allows release of the levosulpiride over a time period of about 6 to 12 hours.

Moreover, the formulation of these pharmaceutical oral dosage forms must result in final products that meet the requirements of regulatory agencies such as the Food and Drug Administration. For example, final products must have a stable product that does not fracture during storage and transport. This is measured for tablets, in part, in terms of friability and hardness. Dosage forms must also meet the requirements for content uniformity, which essentially means that the dispersion of the active ingredient(s) is uniform throughout the mixture used to make the dosage form, such that the composition of tablets formed from a particular formulation does not vary significantly from one tablet to another. The FDA requires a content uniformity within a range of 95% to 105%.

The dosage form as described here comprises one or more swellable polymers and is capable of swelling dimensionally unrestrained in the stomach upon contact with gastric fluid due to the component hydrophilic polymers, for example, polyethylene oxide and/or hypromellose (also known as hydroxypropyl methylcellulose or HPMC), and increase to a size sufficient to be retained in the stomach in a fed mode.

Water-swellable polymers suitable for use herein are those that swell in a dimensionally unrestrained manner upon contact with water Such polymers may also gradually erode over time. Examples of such polymers include polyalkylene oxides, such as polyethylene glycols, particularly high molecular weight polyethylene glycols; cellulose polymers and their derivatives including, but not limited to, hydroxyalkyl celluloses, hydroxymethyl cellulose, hydroxyethyl cellulose, hydroxypropyl cellulose, hydroxypropylmethyl cellulose, carboxymethylcellulose, microcrystalline cellulose; polysaccharides and their derivatives; chitosan; poly(vinyl alcohol); xanthan gum; maleic anhydride copolymers; poly(vinyl pyrrolidone); starch and starch-based polymers; maltodextrins; poly(2-ethyl-2-oxazoline); poly(ethyleneimine); polyurethane; hydrogels; crosslinked polyacrylic acids; and combinations or blends of any of the foregoing.

Further examples are copolymers, including block copolymers and graft polymers. Specific examples of copolymers are PLURONIC® and TECTONIC®, which are polyethylene oxide-polypropylene oxide block copolymers available from BASF Corporation, Chemicals Div., Wyandotte, Mich., USA. Further examples are hydrolyzed starch polyacrylonitrile graft copolymers, commonly known as “Super Slurper” and available from Illinois Corn Growers Association, Bloomington, Ill., USA.

Preferred swellable, erodible hydrophilic polymers suitable for forming the gastric retentive portion of the dosage forms described herein are poly(ethylene oxide), hydroxypropyl methyl cellulose, and combinations of poly(ethylene oxide) and hydroxypropyl methyl cellulose. Poly(ethylene oxide) is used herein to refer to a linear polymer of unsubstituted ethylene oxide. The molecular weight of the poly(ethylene oxide) polymers can range from about 9×10⁵ Daltons to about 8×10⁶ Daltons. A preferred molecular weight poly(ethylene oxide) polymer is about 5×10⁶ Daltons and is commercially available from The Dow Chemical Company (Midland, Mich.) referred to as SENTRY® POLYOX® water-soluble resins, NF (National Formulary) grade WSR Coagulant. The viscosity of a 1% water solution of the polymer at 25° C. preferably ranges from 4500 to 7500 centipoise.

Dosage forms prepared for oral administration according to the present disclosure will generally contain other inactive additives (excipients) such as binders, lubricants, disintegrants, fillers, stabilizers, surfactants, coloring agents, and the like.

Binders are used to impart cohesive qualities to a tablet, and thus ensure that the tablet or tablet layer remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Examples of polyvinylpyrrolidone include povidone, copovidone and crospovidone.

Lubricants are used to facilitate tablet manufacture, promoting powder flow and preventing particle capping (i.e., particle breakage) when pressure is relieved. Useful lubricants are magnesium stearate (in a concentration of from 0.25 wt % to 3 wt %, preferably 0.2 wt % to 1.0 wt %, more preferably about 0.3 wt %), calcium stearate, stearic acid, and hydrogenated vegetable oil (preferably comprised of hydrogenated and refined triglycerides of stearic and palmitic acids at about 1 wt % to 5 wt %, most preferably less than about 2 wt %). Disintegrants are used to facilitate disintegration of the tablet, thereby increasing the erosion rate relative to the dissolution rate, and are generally starches, sodium starch glycolate, croscarmellose sodium, clays, celluloses, algins, gums, or crosslinked polymers (e.g., crosslinked polyvinyl pyrrolidone). Fillers include, for example, materials such as kaolin, powdered cellulose, and microcrystalline cellulose, as well as soluble materials such as mannitol, urea, sucrose, lactose, lactose monohydrate, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions.

The gastric retentive dosage form may be a single layer, bilayer, or multilayer tablet or it may be a capsule. Multilayer tablets include tablets having a shell-and-core configuration in which a core is fully encased by a shell. Tablets may also have a coating with or without the pharmaceutically active agent. The tablet comprises a gastric retentive layer which comprises levosulpiride dispersed in a matrix comprised of at least one hydrophilic polymer which swells upon imbibition of fluid.

In one embodiment, a dosage form is formulated to have a dual-matrix configuration (“shell-and-core”) as described in US publication US 2003/0104062 (herein incorporated by reference). One matrix forms a core of polymeric material in which levosulpiride is dispersed and the other matrix forms a casing that surrounds and fully encases the core, the casing being of polymeric material that swells upon imbibition of water (and hence gastric fluid) to a size large enough to promote retention in the stomach during the fed mode, the shell and core being configured such that the drug contained in the core is released from the dosage form by diffusion through the shell. The shell is sufficient thickness and strength that it is not disrupted by the swelling and remains intact during substantially the entire period of drug release. The shell may or may not contain levosulpiride.

An alternative oral dosage formulation may be the preparation of a tablet which has an immediate release (IR) core containing a drug, completely surrounded or encased by an extended release (ER) shell, wherein the ER shell also contains the drug and swells upon imbibition of fluid (e.g., stomach fluid after administration). The ER shell can be designed as discussed above to rapidly enough to a size sufficient for gastric retention in the stomach of a subject in the fed mode. It is understood that a sufficient size of a tablet for gastric retention may range from about 1 cm to about 15 cm, or at least about 1 cm, 1.5 cm, 2 cm, 2.5 cm, 3 cm, 4 cm or greater.

This alternative “pulsatile release” dosage form may be useful to ensure that levels of the drug released from the dosage form are maintained at therapeutically effective levels. For example, if towards the end of the time period of sustained or extended release of the drug from the ER shell, the drug that has reached the blood of the subject has metabolized or been excreted to a level below that which has optimal therapeutic effect, the subsequence burst of the drug dose from the IR core will bring the drug to a more therapeutic level in the blood. Accordingly, while levosulpiride is an exemplary embodiment of this dosage form (as described later in Example 8), this IR core surrounded by an ER shell which swells and bursts may be applicable to other drugs.

One having ordinary skill in the art would understand that this dosage form may further comprise an IR coat applied to the surface of the ER shell. This IR coat could contain the same drug or a different drug, and the IR coat would dissolve to immediately release the drug into the stomach after oral administration of the tablet.

Water-swellable polymers useful in the preparation of the shell-and-core dosage form include polymers that are non-toxic and, at least in the case of the shell, polymers that swell in a dimensionally unrestricted manner upon imbibition of water. The core polymer may also be a swelling polymer, and if so, compatible polymers will be selected that will swell together without disrupting the integrity of the shell. The core and shell polymers may be the same or different, and if the same, they may vary in molecular weight, crosslinking density, copolymer ratio, or any other parameter that affects the swelling rate, so long as any swelling occurring in the core causes substantially not splitting of the shell.

In one embodiment, a tablet having an immediate release layer encased by an extended release gastric retained layer as a shell is manufactured. The shell-and-core tablet is then spray-coated with an IR layer.

In one embodiment, a tablet is formulated to have an extended release gastric retained (ER) layer spray-coated with an IR layer to provide 12-hour release of levosulpiride. In a further embodiment, the total mass of the tablet is 750 mg and contains 30 mg levosulpiride in the IR layer and 45 mg levosulpiride in the ER layer.

### III. METHODS FOR MAKING THE DOSAGE FORMS

The presently described dosage forms provide for extended release of levosulpiride in the stomach wherein the dosage forms are comprised of a polymer matrix that swells upon imbibition of fluid to a size sufficient for gastric retention. Thus, in formulating the dosage forms, it is desirable to provide the properties which simultaneously allow: a) an extent of swelling to provide gastric retention over an extended period, and b) a rate of swelling and erosion that allows release of the levosulpiride over a time period of about 6 to 12 hours.

Moreover, the formulation of these pharmaceutical oral dosage forms preferably result in final products that meet the requirements of regulatory agencies such as the Food and Drug Administration. For example, final products desirably have a stable product that does not fracture during storage and transport. This is measured for tablets, in part, in terms of friability and hardness. Dosage forms preferably also satisfy requirements for content uniformity, which essentially means that the dispersion of the active ingredient(s) is uniform throughout the mixture used to make the dosage form, such that the composition of tablets formed from a particular formulation does not vary significantly from one tablet to another. The FDA requires a content uniformity within a range of 95% to 105%.

The ability to formulate a pharmaceutical oral dosage form which both delivers the therapeutically effective ingredient over a desired period of time and meets FDA requirements depends, in part and in some embodiments, upon the process by which the product is made.

In the case of gastric retentive tablets containing levosulpiride, as disclosed herein, tablets may be made through direct compression or following a granulation procedure. Direct compression is used with a group of ingredients can be blended, placed onto a tablet press, and made into a perfect tablet without any of the ingredients having to be changed. Powders that can be blended and compressed are commonly referred to as directly compressible or as direct-blend formulations. When powders do not compress correctly, a granulation technique is considered.

Granulation is a manufacturing process which increases the size and homogeneity of active pharmaceutical ingredients and excipients which comprise a solid dose formulation. The granulation process, which is often referred to as agglomeration, changes physical characteristics of the dry formulation, with the aim of improving manufacturability, and therefore, product quality.

Granulation technology can be classified into one of two basic types: wet granulation and dry granulation. Wet granulation is by far the more prevalent agglomeration process utilized within the pharmaceutical industry. Most wet granulation procedures follow some basic steps; the drug(s) and excipients are mixed together, and a binder solution is prepared and added to the powder mixture to form a wet mass. The moist particles are then dried and sized by milling or by screening through a sieve. In some cases, the wet granulation is “wet milled” or sized through screens before the drying step. There are four basic types of wet granulation; high shear granulation, fluid bed granulation, extrusion and spheronization and spray drying.

### A. Dry Granulation

The dry granulation process involves three basic steps; the drug(s) and excipients(s) are mixed (along with a suitable binder if needed) and some form of lubrication, the powder mixture is compressed into dry “compacts,” and then the compacts are sized by a milling step. The two methods by which dry granulation can be accomplished are slugging and roller compaction.

### B. Fluid Bed Granulation

The fluid bed granulation process involves the suspension of particulates within an air stream while a granulation solution is sprayed down onto the fluidized bed. During the process, the particles are gradually wetted as they pass through the spay zone, where they become tacky as a result of the moisture and the presence of binder within the spray solution. These wetted particles come into contact with, and adhere to, other wetted particles resulting in the formation of particles.

A fluid bed granulator consists of a product container into which the dry powders are charged, an expansion chamber which sits directly on top of the product container, a spray gun assembly, which protrudes through the expansion chamber and is directed down onto the product bed, and air handling equipment positioned upstream and downstream from the processing chamber.

The fluidized bed is maintained by a downstream blower which creates negative pressure within the product container/expansion chamber by pulling air through the system. Upstream, the air is “pre-conditioned” to target values for humidity, temperature and dew point, while special product retention screens and filters keep the powder within the fluid bed system.

As the air is drawn through the product retention screen it “lifts” the powder out of the product container and into the expansion chamber. Since the diameter of the expansion chamber is greater than that of the product container, the air velocity becomes lower within the expansion chamber. This design allows for a higher velocity of air to fluidize the powder bed causing the material to enter the spray zone where granulation occurs before loosing velocity and falling back down into the product container. This cycle continues throughout the granulation process.

The fluid bed granulation process can be characterized as having three distinct phases; pre-conditioning, granulation and drying. In the initial phase, the process air is pre-conditioned to achieve target values for temperature and humidity, while by-passing the product container altogether. Once desired or optimal conditions are met, the process air is re-directed to flow through the product container, and the process air volume is adjusted to a level which will maintain sufficient fluidization of the powder bed. This pre-conditioning phase completes when the product bed temperature is within the target range specified for the process.

In the next phase of the process, the spraying of the granulating solution begins. The spray rate is set to a fall within a pre-determined range, and the process continues until all of the solution has been sprayed into the batch. It is in this phase where the actual granulation, or agglomeration, takes place.

Once the binder solution is exhausted, the product continues to be fluidized with warm process air until the desired end-point for moisture content is reached. This end-point often correlates well with product bed temperature, therefore in a manufacturing environment, the process can usually be terminated once the target product bed temperature is reached. A typical fluid bed process may require only about thirty to forty-five minutes for the granulation step, plus ten to fifteen minutes on either side for pre-conditioning and drying.

As with any of the wet granulation processes, a variable is the amount of moisture required to achieve successful agglomeration. The fluid bed granulation process preferably provides a “thermodynamic” balance between process air temperature, process air humidity, process air volume and granulation spray rate. While higher process air temperature and process air volume add more heat to the system and remove moisture, more granulating solution and a higher solution spray rate add moisture and remove heat via evaporative cooling. These are process parameters which are preferably evaluated as a manufacturing process is developed, and a key is understanding the interdependency of each variable.

Additional factors affecting the outcome of the fluid bed granulation process are the amount and type of binder solution, and the method by which the binder is incorporated within the granulation. Other process variables are the total amount of moisture added through the process, and the rate at which the moisture content is increased. These parameters can have an effect on the quality and the characteristics of the granulation. For instance, a wetter fluid bed granulation process tends to result in a stronger granule with a higher bulk density. However, an overly aggressive process, where moisture is added too rapidly, can loose control over achieving the final particle size and particle size distribution objectives.

### C. High Shear Granulation

Most pharmaceutical products manufactured by wet granulation utilize a high shear process, where blending and wet massing are accomplished by the mechanical energy generated by an impeller and a chopper. Mixing, densification and agglomeration are achieved through the “shear” forces exerted by the impeller; hence the process is referred to as high shear granulation.

The process begins by adding the dry powders of the formulation to the high shear granulator, which is a sealed “mixing bowl” with an impellor which rotates through the powder bed, and a chopper blade which breaks up over-agglomerates which can form during the process. There are typically three phases to the high shear process; dry mixing, solution addition, or wet massing and high shear granulation.

In the first phase, dry powders are mixed together by the impeller blade which rotates through the powder bed. The impeller blade is positioned just off the bottom of the product container. There is a similar tolerance between the tips of the impeller blade and the sides of the container. The impeller blades rotation trough the powder bed creates a “roping” vortex of powder movement. The dry mixing phase typically lasts for only a few minutes.

In the second phase of the process, a granulating liquid is added to the sealed product container, usually by use of a peristaltic pump. The solution most often contains a binder with sufficient viscosity to cause the wet massed particles to stick together or agglomerate. It is common for the solution addition phase to last over a period of from three to five minutes. While the impeller is rotating rather slowly during this step of the process, the chopper blade is turning at a fairly high rate of speed, and is positioned within the product container to chop up over-sized agglomerates, while not interfering with the impellers movement.

Once the binder solution has been added to the product container, the final stage of the granulation process begins. In this phase, high shear forces are generated as the impeller blades push through the wet massed powder bed, further distributing the binder and intimately mixing the ingredients contained therein. The impeller and chopper tool continue to rotate until the process is discontinued when the desired granule particle size and density end-points are reached. This end-point is often determined by the power consumption and/or torque on the impeller.

Once the high shear granulation process has been completed, the material is transferred to a fluid bed dryer, or alternatively, spread out onto trays which are then placed in a drying oven, where the product is dried until the desired moisture content is achieved, usually on the order of 1-2% as measured by Loss On Drying technique.

A variable which affects the high shear process is the amount of moisture required to achieve a successful granulation. A key to the process is having the right amount of moisture to allow for agglomeration to occur. Too little moisture will result in an under-granulated batch, with weak bonds between particles and smaller, to non-existent particles, with properties similar to those of the dry powder starting materials. On the other hand, excess moisture can result in a “crashed” batch with results varying from severe over-agglomeration to a batch which appears more like soup.

Other formulation parameters affecting the outcome of the high shear granulation process are the amount and type of binder solution, and the method by which the binder is incorporated within the granulation. For example, it is possible to include some of the binder in the dry powder mixture as well as in the granulating solution, or it may be incorporated only in the granulating solution or only in the dry powder, as is the case where water is used as the granulating solution.

The high shear granulation process parameters which are variable include impeller and chopper speeds, the solution addition rate, and the amount of time allocated to the various phases of the process. Of these, variables for consideration are the solution addition rate and the amount of time the wet massed product is under high shear mixing

### D. Extrusion and Spheronization

This specialized wet granulation technique involves multiple processing steps and was developed to produce very uniform, spherical particles ideally suited for multi-particulate drug delivery of delayed and sustained release dosage forms.

Similar to high shear granulation initially, the first step involves the mixing and wet massing of the formulation. Once this step is complete, the wet particles are transferred to an extruder which generates very high forces used to press the material out through small holes in the extruder head. The extrudate is of uniform diameter and is then transferred onto a rotating plate for spheronization. The forces generated by the rotating plate initially break up the extruded formulation strands into uniform lengths. Additional dwell time within the spheronizer creates particles which are quite round and very uniform in size. These pellets or spheres must then be dried to the target moisture content, usually within a fluid bed system.

Particles produced in this manner tend to be very dense, and have a capacity for high drug loading, approaching 90% or more in some cases. Preferably, particle size is uniform, and the size distribution is narrow, as compared to other granulation approaches. This quality assures consistent surface area within and between batches, which is desired when functional coatings are subsequently applied to create sustained release formulations, delayed release formulations and formulations designed to target a specific area within the body.

Uniform surface area is desired because the pharmaceutical coating process endpoint is determined not by coating thickness, but by the theoretical batch weight gain of the coating material. If the batch surface area is consistent, then the coating thickness will also be consistent for a given weight gain, and coating thickness is the primary variable in determining the functionality of the coating system, whether the goal is controlling the duration of sustained release formulations or imparting an acid resistant characteristic to “beads” necessary to protect certain compounds which would otherwise be severely degraded in the presence of the acidic environment of the stomach.

### E. Spray Drying

Spray drying is a unique and specialized process which converts liquids into dry powders. The process involves the spraying of very finely atomized droplets of solution into a “bed” or stream of hot process air or other suitable gas. Not typically utilized for the conventional granulation of dosage form intermediates, spray drying has gained acceptance within the industry as a robust process which can improve drug solubility and bioavailability.

Spray drying can be used to create co-precipitates of a drug/carrier which can have improved dissolution and solubility characteristics. In addition, the process can also be useful as a processing aid. For example, it is much more difficult to maintain the uniformity of a drug in suspension, as compared to the same compound in solution. One may have a need to develop an aqueous coating or drug layering process utilizing a drug which is otherwise not soluble in water. By creating a co-precipitate of the drug and a suitable water soluble carrier, often a low molecular weight polymer, the co-precipitate will remain in solution throughout the manufacturing process, improving uniformity of the spray solution and the dosage form created by the coating process. Uniformity is particularly desired where lower doses of potent compounds are intended to be coated onto beads or tablet cores.

This same process may be used to enhance the solubility and bioavailability of poorly soluble drugs. By complexing certain excipients and the active ingredient within a solvent system which is then spray dried, it is possible to enhance the drugs absorption within the body. Selection of the solvent system, the complexing agent(s) and the ratios utilized within the formulation are all formulation variables which determine the effectiveness of solubility enhancement utilizing the spray drying technique. Process parameters which also have an effect on drug solubility are the temperatures of the spray solution and process gas, the spray rate and droplet size and the rate of re-crystallization. The spray dried granulations created by these techniques can then be incorporated into capsules or tablets by conventional manufacturing processes.

### IV. METHODS OF MAKING THE EXTENDED RELEASE GASTRIC RETENTIVE DOSAGE FORMS DISCLOSED HEREIN

In one aspect, a method of making a gastric retentive extended-release dosage form as a single layer tablet comprising dry blending of the levosulpiride with the binder is provided. The blended material is then granulated in the presence of water using, for example, a KitchenAid® blender. The granulated particles are then dried overnight, screened and blended with additional excipients as needed to form a mixture which is then compressed to form tablets.

Extended release polymer matrices comprising levosulpiride are made using either POLYOX® 1105 (approximate molecular weight of 900,000 Daltons), POLYOX® N-60K (approximate molecular weight of 2,000,000 Daltons), or POLYOX® WSR-301 (approximate molecular weight of 4,000,000 Daltons).

After granulation of the active ingredient and subsequent blending with additional excipients, batches are characterized with respect to properties such as final Loss on Drying (LOD), bulk density, tap density, and particle size.

Loss on Drying (LOD) is determined after each granulation using the Moisture Analyzer. A 1 g samples are taken and loaded into the moisture analyzer. The sample is run for 5 minutes at a temperature of 105° C.

Bulk and tap densities can be determined as follows. A graduated cylinder is filled with a certain amount of material (82-88 g), and the volume recorded to determine the material bulk density. Tap density can be determined with a help of a Tap Density Tester by exposing the material to 100 taps per test and recording the new volume.

Particle size determination is performed immediately after granulation, after sieving through 20 mesh screen to remove agglomerates. Particle diameter is determined with a sieve-type particle diameter distribution gauge using sieves with openings of 44, 53, 75, 106, 150, and 250 mesh. Fractions are weighed on Mettler balance to estimate size distribution. This provides determination of the quantitative ratio by particle diameter of composition comprising extended release particles. Sieve analysis according to standard United States Pharmacopoeia methods (e.g., USP-23 NF 18), may be done such as by using a Meinzer II Sieve Shaker.

The granulated mixture can be blended with the polymer, filler and lubricant in a V-blender. The resultant mixture can be compressed into monolithic, single-layer tablets using, for example, a Piccola Press or a Manesty® BB4 press, with the appropriate tooling.

Tablets may then be characterized with respect to disintegration and dissolution release profiles as well as tablet hardness, friability and content uniformity.

The dissolution profiles for the tablets may be determined in a USP apparatus III (disintegration apparatus), 30 dpm (dips per minute), in 0.1 N HCl at 37° C. Samples of 5 ml at each time-point, may be taken without media replacement at 1, 2, 4, 6, 8 and 12 hours. The resulting cumulative dissolution profiles for the tablets are based upon a theoretical percent active added to the formulations.

A tablet must disintegrate before it dissolves. A disintegration tester measures the time it takes a tablet to break apart in solution. The tester suspends tablets in a solution bath for visual monitoring of the disintegration rate. Both the time to disintegration and the disintegration consistency of all tablets are measured. The disintegration profile is determined in a USP Disintegration Tester in 0.1N HCl at 37° C. Samples, 1 ml at each time-point, may be taken, for example, without media replacement at 0.5, 1, 2, 3, 4, 5, 6, 7 and 8 hours. The resulting cumulative disintegration profiles are based upon a theoretical percent active added to the formulation is determined.

Tablet hardness changes rapidly after compression as the tablet cools. A tablet that is too hard may not break up and dissolve into solution before it passes through the body. In the case of the presently disclosed gastric retentive dosage forms, a tablet that is too hard may not be able to imbibe fluid rapidly enough to prevent passage through the pylorus in a stomach in a fed mode. A tablet that is too soft may break apart, not handle well, and can create other defects in manufacturing. A soft tablet may not package well or may not stay together in transit.

After tablets are formed by compression, it is desired that the tablets have a strength of at least 9-25 Kiloponds (Kp) preferably at least about 12-20 (Kp). A hardness tester is used to determine the load required to diametrically break the tablets (crushing strength) into two equal halves. The fracture force may be measured using a Venkel Tablet Hardness Tester, using standard USP protocols.

Friability is a well-known measure of a tablet's resistance to surface abrasion that measures weight loss in percentage after subjecting the tablets to a standardized agitation procedure. Friability properties are especially relevant during any transport of the dosage form as any fracturing of the final dosage form will result in a subject receiving less than the prescribed medication. Friability can be determined using a Roche Friability Drum according to standard USP guidelines which specifies the number of samples, the total number of drum revolutions and the drum rpm to be used. Friability values of from 0.8 to 1.0% are regarded as constituting the upper limit of acceptability.

The prepared tablets may be tested for content uniformity to determine if they meet the pharmaceutical requirement of <6% relative standard deviation (RSD). Each tablet is placed in a solution of 1.0 N HCl and stirred at room temperature until all fragments have visibly dissolved. The solution containing the dissolved tablet is analyzed by HPLC.

In one aspect, a method of making a bilayer tablet comprising a gastric retentive extended-release layer and an immediate release layer is provided.

### V. STABILITY OF LEVOSULPIRIDE EXTENDED RELEASE FORMULATIONS

Stability testing is the primary tool used to assess expiration dating and storage conditions for pharmaceutical products. Many protocols have been used for stability testing, but most in the industry are now standardizing on the recommendations of the International Conference on Harmonization (ICH). These guidelines were developed as a cooperative effort between regulatory agencies and industry officials from Europe, Japan, and the United States.

Stability testing includes long-term studies, where the product is stored at room temperature and humidity conditions, as well as accelerated studies where the product is stored under conditions of high heat and humidity. Proper design, implementation, monitoring and evaluation of the studies are crucial for obtaining useful and accurate stability data. Stability studies are linked to the establishment and assurance of safety, quality and efficacy of the drug product from early phase development through the lifecycle of the drug product. Stability data for the drug substance are used to determine optimal storage and packaging conditions for bulk lots of the material. The stability studies for the drug product are designed to determine the expiration date (or shelf life). In order to assess stability, the appropriate physical, chemical, biological and microbiological testing must be performed. Usually this testing is a subset of the release testing.

Studies are designed to degrade the solid drug substance and appropriate solutions, allowing the determination of the degradation profile. The drug substance is usually challenged under a variety of accelerated environmental conditions to evaluate its intrinsic stability and degradation profile.

HPLC is the predominant tool used to analyze the drug substance and the impurities, particularly for small molecules. Frequently, the same HPLC method may be used for drug substance and drug product, although different sample preparation methods would normally be required. Often the assay and impurity testing can be performed using a single HPLC method. However, the assay and purity determinations may also be separate methods. At least in the U.S., full validation of the analytical method is not required until the end of Phase 2 clinical trials, but the establishment of specificity, linearity and limit of quantification (for impurities) is considered at the earliest stages, since verification of stability hinges on a suitable method for separating impurities from the active ingredient and at least quantifying the impurities relative to the drug substance.

Stress studies at elevated temperature (e.g., 50° C., 60° C. and 70° C.) for several weeks may be performed to assess thermal stability. Provided the degradation mechanism is the same at the different temperatures used, kinetic or statistical models can be used to determine the rate of degradation at other temperatures (e.g., 25° C.). The solid stability should also be performed in the presence and absence of water vapor to assess the dependence of stability on humidity.

Degradation studies should also be performed in solution. The solvent used for the solution testing will depend on the solubility of the drug substance and should include water, if the drug substance is water-soluble. Other solutions or solvent systems may be evaluated depending on the anticipated formulation or the synthetic process. A series of buffered solutions in the pH range 2-9 are useful in assessing the impact of solution pH on the degradation. Photostability should also be evaluated. A xenon light source can be used as a stress condition. Alternatively, one can use an accelerated version of either Options 1 or 2 as described in the ICH guideline for determination of photostability. Oxidation of the drug substance under accelerated conditions (e.g., hydrogen peroxide), may also be performed to establish oxidation products that could be formed and sensitivity to oxidative attack.

Early drug product stability studies are designed to help establish a suitable formulation for delivery of the drug substance. Compatibility studies of the drug substance with excipients should be performed to eliminate excipients that are not compatible with the drug substance.

### VI. METHODS OF TREATMENT

In another aspect, the dosage form comprising levosulpiride is administered to a subject suffering from, for example, dyspeptic syndrome (anorexia, meterioam, sense of epigastric tension, postprandial cephalgia, pyrosis, eruptions, diarrhea, stipsis) due to retarded gastric voiding related to organic factors (diabetic gastroperesis, neoplasia, etc.) and/or function factors (visceral somatization in anxious, depressive individual), vomiting and/or nausea (post-operatorial or induced by chemotherapy, gastroesophageal reflux disease (GERD) and irritable bowel disorder (IBD). In another embodiment, the dosage formulations comprising levosulpiride as disclosed herein may be used to treat schizophrenia.

Generally, the frequency of administration of a particular dosage form is determined to provide the most effective results in an efficient manner without overdosing and varies according to the following criteria: (1) the characteristics of the particular drug(s), including both its pharmacological characteristics and its physical characteristics, such as solubility; (2) the characteristics of the swellable matrix, such as its permeability; and (3) the relative amounts of the drug and polymer. In most cases, the dosage form is prepared such that effective results are achieved with administration once every six hours, once every eight hours, once every twelve hours, or once every twenty-four hours. As previously discussed, due to the physical constraints placed on a tablet or capsule that is to be swallowed by a patient, most dosage forms can only support a limited amount of drug within a single dosage unit.

In one embodiment, the dosage form allows a dosing frequency of two times a day (b.i.d.) or once per day (q.d.) to result in sustained plasma concentration of the drug as compared to current immediate release products which require more frequent administration for effective sustained relief.

Within the context of the present disclosure, the gastric retentive dosage forms have the advantage of improving patient compliance with administration protocols because the drugs may be administered in a once-daily or twice-daily dosing regimen, rather than the multiple dosing administrations necessary for the immediate release dosage forms of levosulpiride in order to maintain a desired level of treatment. One embodiment of the invention relates to a method of administering a therapeutically effective amount of a levosulpiride to a patient in need thereof, comprising administering the levosulpiride or pharmaceutically acceptable salts thereof, in a gastric retentive dosage form once in the morning or evening in a once a day daily regime. Another embodiment comprises administering the gastric retentive dosage form twice a day, for example once in the morning and once in the evening in a twice a day daily dosage regime.

For all modes of administration, the gastric retentive dosage forms described herein are preferably administered in the fed mode, i.e., with or just after consumption of a small meal (see U.S. Publication No. 2003/0104062, herein incorporated by reference). When administered in the evening fed mode, the gastric retentive dosage form may provide the subject with continued relief from the symptoms of, for example, dyspepsia or GERD. The gastric retentive dosage form as presented herein allows for both extended release of the levosulpiride and the superior absorption of the drugs in the GI tract.

In some aspects, the postprandial or fed mode can also be induced pharmacologically, by the administration of pharmacological agents that have an effect that is the same or similar to that of a meal. These fed-mode inducing agents may be administered separately or they may be included in the dosage form as an ingredient dispersed in the shell, in both the shell and the core, or in an outer immediate release coating. Examples of pharmacological fed-mode inducing agents are disclosed in U.S. Pat. No. 7,405,238, entitled “Pharmacological Inducement of the Fed Mode for Enhanced Drug Administration to the Stomach,” inventors Markey, Shell, and Berner, the contents of which are incorporated herein by reference.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the present disclosure or its claims.

### EXAMPLES

The following examples are intended to illustrate the dosage forms, methods of manufacture, and methods of treatment, and are not intended to limit the disclosure.

### Example 1

### Preparation of a Gastric Retentive Dosage Form Having 75 mg Levosulpiride

An extended release gastric retentive oral dosage form containing 75 mg levosulpiride was formulated to provide 6-hour, 8-hour and 12-hour release. Tablets having a total mass of 700 mg were manufactured. A 5% solution of povidone in water was prepared and granulated using a KitchenAid® blender with the levosulpiride. Granules were dried in a 50±5° C. oven overnight, then granules were screened through a #30 mesh US standard screen. Screened levosulpiride-containing granules were then blended with the remaining excipients. Tablets were compressed by a manual hand crank mode on a Piccola Press, tooled with a 0.3937″×0.6299″ die. Tablets had a hardness of 13-15 Kiloponds (Kp). Amounts of levosulpiride, binder and excipients for each formulation are listed in Table 1 below.

TABLE 1

| | Weight % | | |
|---|---|---|---|
| | 6-hour | 8-hour | 12-hour |
| Ingredients | release tablet | release tablet | release tablet |
| Levosulpiride | 10.71 | 10.71 | 10.71 |
| Povidone K29/32 | 0.56 | 0.56 | 0.56 |
| Polyox 1105, LEO NF | 50 | 87.72 | — |
| Polyox N60K | — | — | 80 |
| Microcrystalline | 37.72 | — | 7.72 |
| Cellulose (MCC) PH- | | | |
| 101 | | | |
| Magnesium Stearate | 1 | 1 | 1 |
| Total wt % | 100 | 100 | 100 |

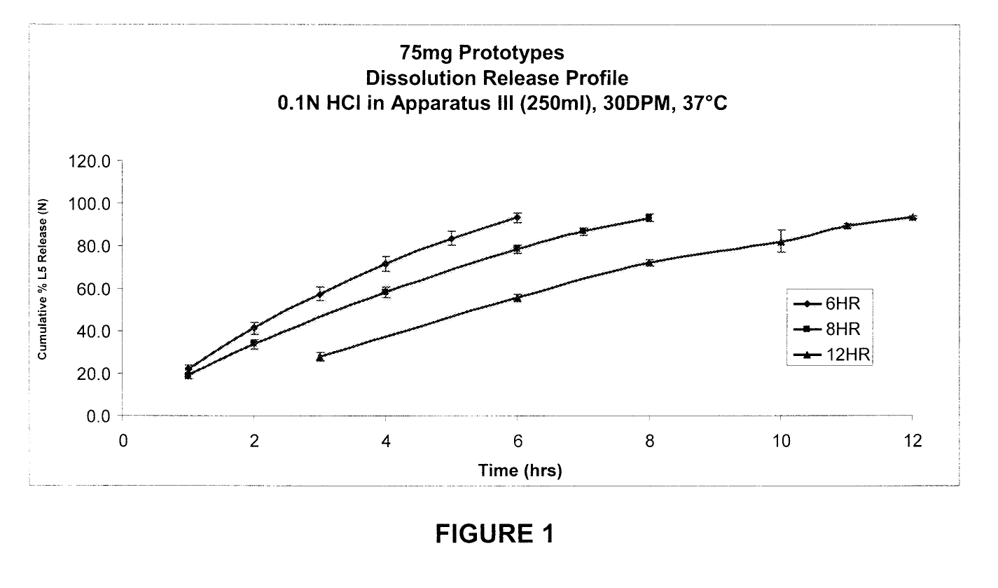

Dissolution release profiles for the tablets produced above were determined in an Apparatus III (250 ml) USP Disintegration Tester in 0.1 N HCl, 30 dpm (dips per minute) at 37±2° C. Results are presented below in Table 2 and graphically in FIG. 1 and show cumulative release of about 93% of the levosulpiride in the 6-hour, 8-hour and 12-hour release tablets by 6, 8, and 12 hours, respectively.

TABLE 2

| | Cumulative Release (%) | | |
|---|---|---|---|
| Time (hours) | 6-Hour Tablet | 8-Hour Tablet | 12-Hour Tablet |
| 1 | 21.7 | 18.9 | — |
| 2 | 41.1 | 33.8 | — |
| 3 | 57.4 | — | 27.8 |
| 4 | 71.8 | 58.3 | — |
| 5 | 83.7 | — | — |
| 6 | 93.4 | 78.6 | 55.6 |
| 7 | — | 86.9 | — |
| 8 | — | 93.3 | 72.0 |
| 10 | — | — | 82.4 |
| 11 | — | — | 89.7 |
| 12 | — | — | 93.7 |

### Example 2

### Preparation of a Gastric Retentive Dosage Form Having 37.5 mg Levosulpiride

An extended release gastric retentive oral dosage form containing 37.5 mg levosulpiride was formulated to provide 6-hour and 8-hour release. Tablets having a total mass of 700 mg were manufactured using a wet granulation process followed by screening, blending and compression. A 5% solution of povidone in water was prepared and blended with the levosulpiride. The material was granulated using a KitchenAid® blender. The wet granules were then dried in a 50±5° C. oven overnight, then granules were screened through a #30 mesh US standard screen. Screened active granules were then blended with the remaining excipients. Tablets were compressed by a manual hand crank mode on a Piccola Press, tooled with a 0.3937″×0.6299″ die. Tablets had a hardness of 13-15 Kp. Amounts of levosulpiride, binder and excipients for each formulation are listed in Table 3 below.

TABLE 3

Weight %

| | 6-hour | 8-hour |
|---|---|---|
| Ingredients | release tablet | release tablet |
| Levosulpiride | 5.36 | 5.36 |
| Povidone K29/32 | 0.85 | 0.85 |
| Polyox 1105, LEO NF | 50 | 82.08 |
| Microcrystalline | 42.79 | 10.71 |
| Cellulose (MCC) PH- | | |
| 101 | | |
| Magnesium Stearate | 1 | 1 |
| Total wt % | 100 | 100 |

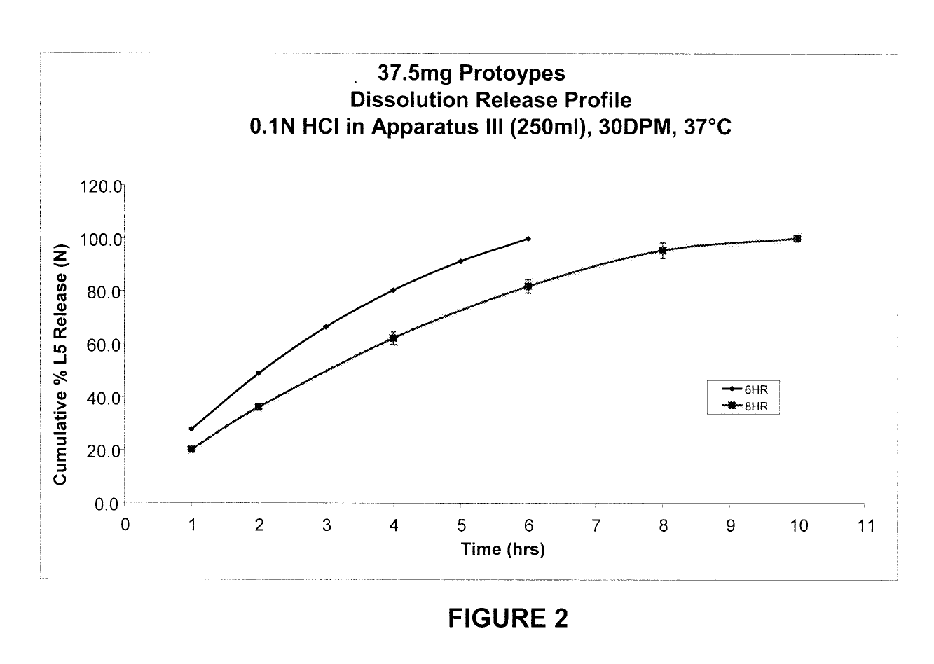

Dissolution release profiles for the tablets produced above were determined in an Apparatus III (250 ml) USP Disintegration Tester in 0.1 N HCl, 30 dpm (dips per minute) at 37±2° C. Results are presented below in Table 4 and graphically in FIG. 2 and show cumulative release of 100% of the levosulpiride in the 6-hour and 95% in the 8-hour release tablets by 6 and 8 hours, respectively.

TABLE 4

Cumulative Release (%)

| Time (hours) | 6-Hour Tablet | 8-Hour Tablet |
|---|---|---|
| 1 | 27.8 | 20.0 |
| 2 | 48.9 | 36.1 |
| 3 | 66.4 | — |
| 4 | 80.4 | 62.2 |
| 5 | 91.4 | — |
| 6 | 100.0 | 81.8 |
| 7 | — | — |
| 8 | — | 95.5 |
| 10 | — | 100.0 |

### Example 3

### Formulation of a 75 Mg and a 37.5 mg IR/ER Bilayer Tablet

Bilayer tablets having an immediate release (IR) layer and an extended release (ER) gastric retained layer were formulated to provide 12-hour release. In one formulation, shown in Table 5, the total mass of the tablet was 1000 mg and contained 30 mg levosulpiride in the IR layer and 45 mg levosulpiride in the GR layer. In a second formulation, shown in Table 6, the total mass of the tablet was 1000 mg and contained 6.5 mg levosulpiride in the IR layer and 31 mg levosulpiride in the ER layer.

TABLE 5

| | |
|---|---|
| IR Layer | ER Layer |

| Ingredient | Mg | wt % | Mg | wt % |
|---|---|---|---|---|
| Levosulpiride | 30 | 10 | 45 | 6.43 |
| Microcrystalline | 171.71 | 57.24 | 85.63 | 12.23 |
| cellulose PH-101 | | | | |
| Lactose | 85.88 | 28.63 | — | — |
| Povidone K29/32 | 5.17 | 1.72 | 2.37 | 0.34 |
| Ac-Di-Sol | 3.11 | 1.04 | — | — |
| Crospovidone XL | 2.07 | 0.69 | — | — |
| Polyox N60K, | — | — | 560 | 80 |
| PEO NF | | | | |
| Opadry Blue | 1.03 | 0.34 | — | — |
| Mg Stearate | 1.03 | 0.34 | 7 | 1 |
| Total | 300.0 | 100.0 | 700.0 | 100.0 |

TABLE 6

| | |
|---|---|
| IR Layer | ER Layer |

| Ingredient | Mg | wt % | Mg | wt % |
|---|---|---|---|---|
| Levosulpiride | 6.5 | 2.17 | 31 | 4.43 |
| Microcrystalline | 184.6 | 61.53 | 100.38 | 14.34 |
| cellulose PH-101 | | | | |
| Lactose | 92.33 | 30.78 | — | — |
| Povidone K29/32 | 6.91 | 2.3 | 1.65 | 0.24 |
| Ac-Di-Sol | 4.15 | 1.38 | — | — |
| Crospovidone XL | 2.76 | 0.92 | — | — |
| Polyox N60K, | — | — | 560 | 80 |
| PEO NF | | | | |
| Opadry Blue | 1.38 | 0.46 | 7 | 1 |
| Magnesium | 1.38 | 0.46 | 7 | 1 |
| Stearate | | | | |
| Total | 300.0 | 100.0 | 700.0 | 100.0 |

To produce the IR layer, the levosulpiride (Think Chemicals, China), microcrystalline cellulose, lactose, povidone and croscarmellose sodium (Ac-Di-Sol) was dry blended then granulated in a KitchenAid® blender using water. The granules were dried overnight in an oven at 50±5° C. then screened through a #30 mesh screen. The screened granules were then blended with the remaining excipients (crospovidone, Opadry blue and Magnesium stearate).

To produce the ER layer, the levosulpiride was granulated with a 5% solution of povidone in water in a KitchenAid® blender. The granules were dried overnight in an oven at 50±5° C. then screened through a #30 mesh screen. The screened granules were then blended with the remaining excipients (Polyox N60K, Opadry blue and magnesium stearate).

To manufacture the bilayer tablets, 300 mg of the IR blend was placed into a tooling die (0.3937″×0.7086″) then lightly tapped with the upper punch. Seven hundred mg of the GR blend was then poured into the tooling die. With the upper punch in place, the bilayer tablet was compressed using a single-punch tablet press with 1000 lbs force, 100% pump speed and 0 seconds dwell time. Bilayer tablets containing a total of 37.5 mg levosulpiride had a thickness of 8.2 mm and a hardness of 21.2 Kp.

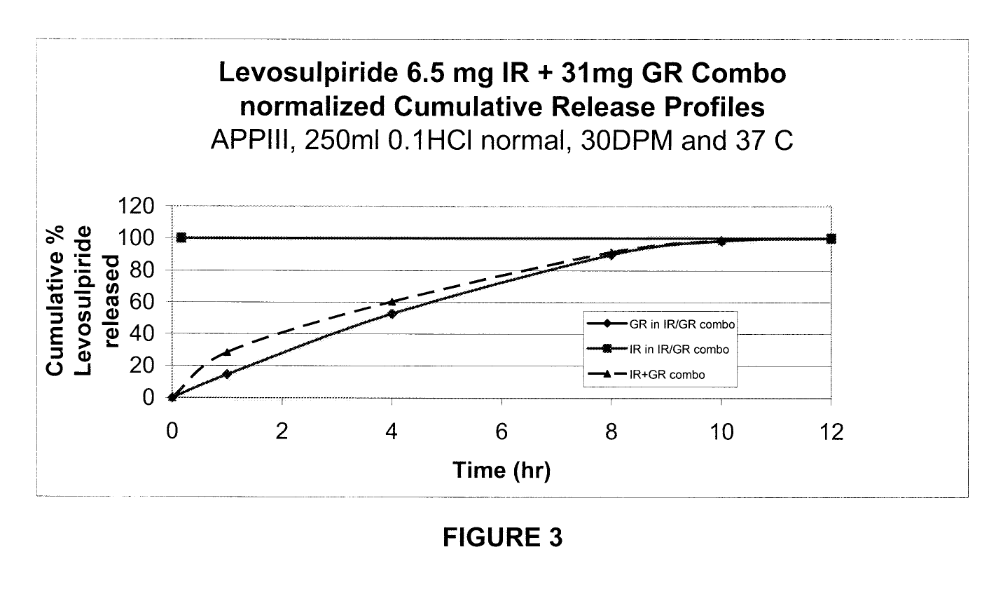

Dissolution release profiles for the bilayer tablets containing a total of 37.5 mg levosulpiride produced as described above were determined in an Apparatus III (250 ml) USP Disintegration Tester in 0.1 N HCl, 30 dpm (dips per minute) at 37±2° C. Results are presented below in Table 7 and graphically in FIG. 3, and show cumulative release of essentially all of the levosulpiride with 12 hours.

TABLE 7

Cumulative Release (%)

| Time (hours) | GR in IR/GR Combo | IR + GR Combo |
|---|---|---|
| 1 | 14.33 | 28.32 |
| 4 | 52.70 | 60.43 |
| 8 | 89.70 | 91.4 |
| 10 | 98.38 | 98.65 |
| 12 | 100 | 100 |

Bilayer tablets are also manufactured using a Manesty® BB4 press.

### Example 4

### Dissolution Studies at Various pH

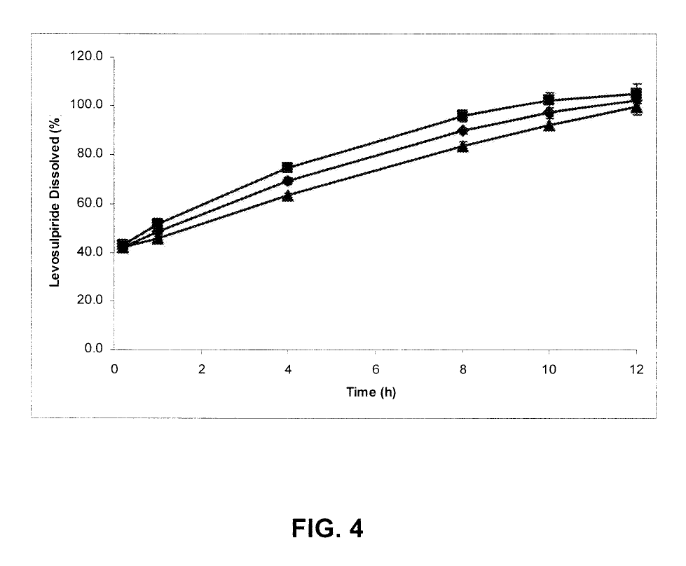

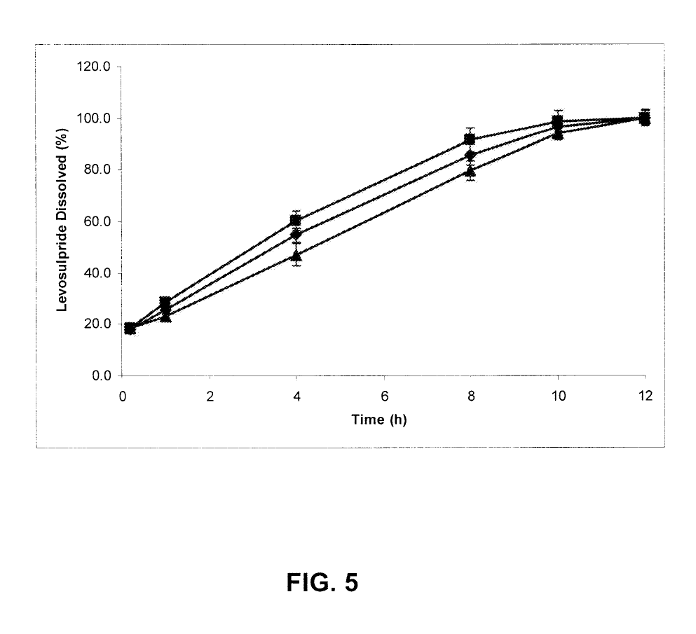

Dissolution rate studies were performed in media of varied pH, including mSGF (modified simulated gastric fluid), acetate buffer of pH 4.5 and phosphate buffer of pH −6.8, 250 ml, at 37±0.2° C., using USP Apparatus III with reciprocating vessels. The experiments were carried out using 6 tablets at each pH The average cumulative drug released with standard deviation was determined and results are presented in Tables 8 and 9. Graphical representations of Tables 8 and 9 are shown in FIGS. 4 and 5, respectively. Table 8 shows dissolution data for the 75 mg bilayer tablet. Table 9 shows dissolution data for the 37.5 mg bilayer tablet. Data in Table 9 are normalized such that 100.0% of the levosulpiride is released by the tablets at each pH at the 12 hours time point.

TABLE 8

| | Cumulative Release (%) | | |
|---|---|---|---|
| Time (hours) | pH 1.2 | pH 4.5 | pH 6.8 |
| 0.2 | 43.0 | 42.1 | 42.0 |
| 1.0 | 51.4 | 48.3 | 45.9 |
| 4.0 | 74.7 | 69.2 | 63.2 |
| 8.0 | 96.1 | 89.9 | 84.0 |
| 10.0 | 102.2 | 97.5 | 92.6 |
| 12.0 | 105.0 | 102.5 | 99.5 |

TABLE 9

| | Cumulative Release (%) | | |
|---|---|---|---|
| Time (hours) | pH 1.2 | pH 4.5 | pH 6.8 |
| 0.2 | 18.3 | 17.9 | 18.5 |
| 1.0 | 28.3 | 25.7 | 22.9 |
| 4.0 | 60.4 | 54.9 | 47.1 |
| 8.0 | 91.4 | 85.8 | 79.7 |
| 10.0 | 98.7 | 96.3 | 93.9 |
| 12.0 | 100.0 | 100.0 | 100.0 |

The in vitro drug release profiles differed at the different pH levels.

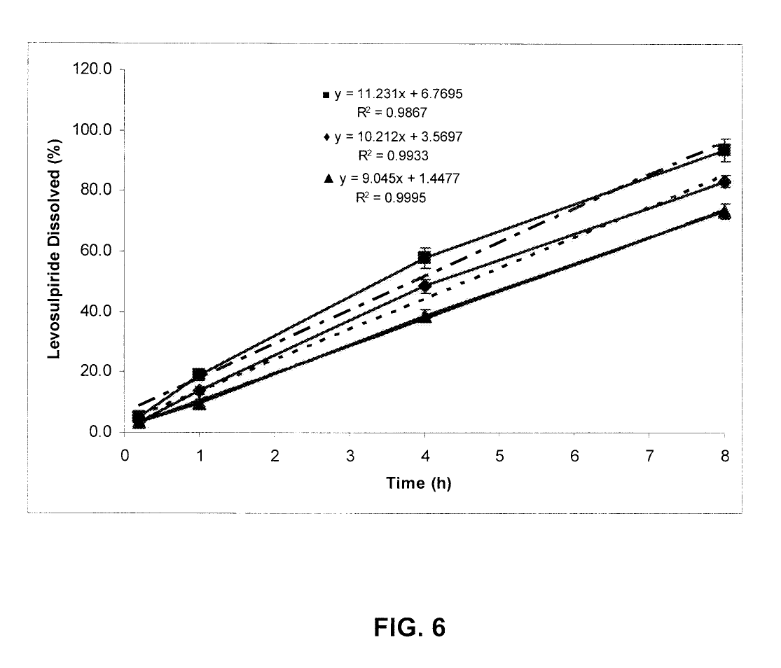

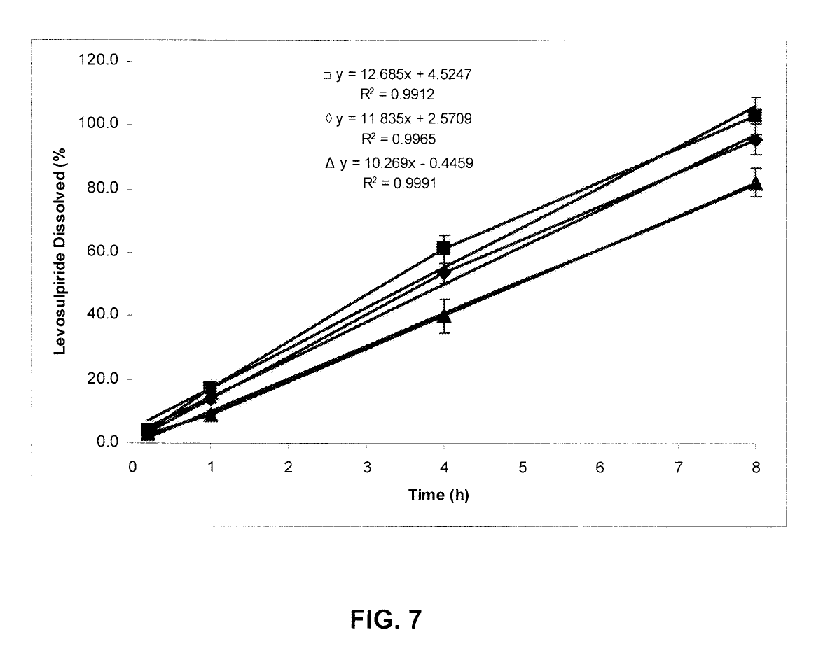

The in vitro drug release data show that the levosupiride bilayer formulations can deliver the drug for about 8 to 12 hours at a release rate of near zero order (FIGS. 5 and 6; ▪ pH 1.2; ♦ pH 4.5; ▴ pH 6.8) Furthermore, erosion is the primary mechanism of drug release from the ER layer as demonstrated by linear regression of the percentage of drug release as a function of time plot, shown in FIGS. 6 and 7 (▪ pH 1.2; ♦ pH 4.5; ▴ pH 6.8), for the 75 mg and 37.5 mg bilayer tablets, respectively. The erosional release of levosulpiride from the bilayer tablets is primarily due to the poor water solubility of the levosulpiride.

### Example 5

### Pharmacokinetic Simulation

Pharmacokinetic simulation was performed for the once-daily 75 mg bilayer dosage form and for the twice-daily 37.5 mg bilayer dosage form to compare plasma concentrations of levosulpiride provided by the gastric retentive dosage forms to that provided by the commercial immediate release product containing 25 mg levosulpiride (LEVOPRAID® 25, Pacific Pharmaceuticals, Pakistan) administered three-times daily. The simulations were carried out using the WINNONLIN® software (Pharsight, St. Louis, Mo.).

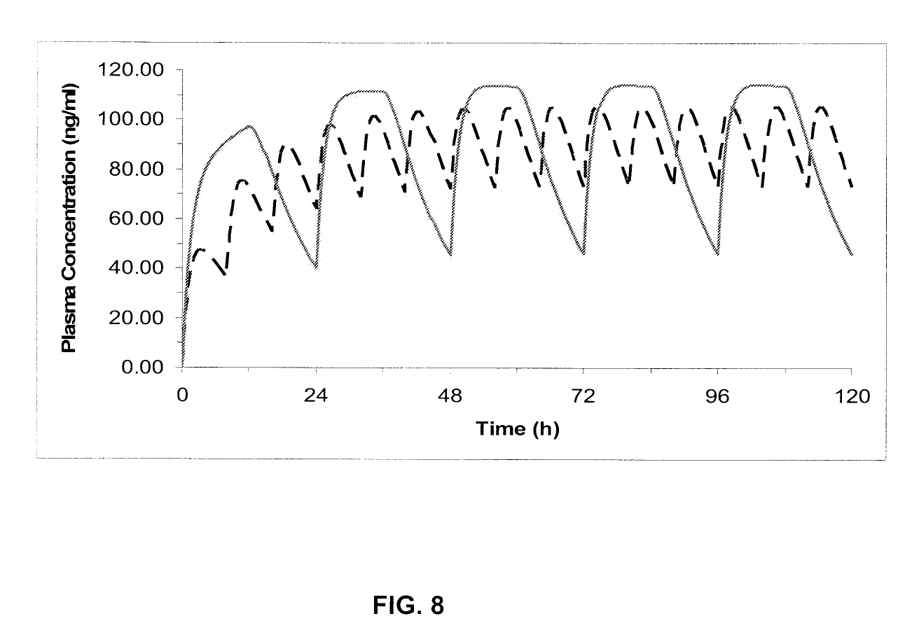

FIG. 8, which depicts the simulated plasma concentration provided by the 75 mg levosulpiride q.d. formulation as compared to the t.i.d. immediate release commercial product shows that at steady state, the dual release gastric retentive formulation with q.d. dosing (solid line) exhibits comparable average plasma concentration to the commercial immediate release product with t.i.d. dosing (dashed line). However, a higher peak-to-trough ratio was observed.

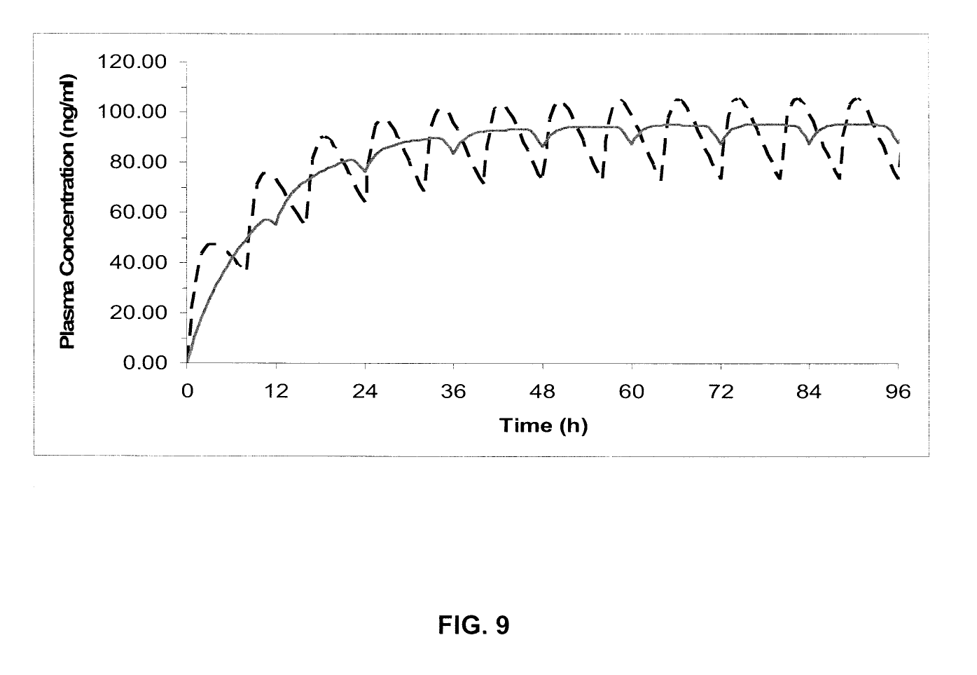

PK simulation comparing b.i.d. dosing with the 37.5 mg formulation to the t.i.d. immediate release commercial product shows that at steady state the dual release gastric retentive formulation with b.i.d. dosing (solid line) exhibits comparable average plasma concentration to the commercial immediate release product with t.i.d. dosing (dashed line). A less variable plasma profile with a lower peak-to-trough ratio was observed for twice daily formulation, as compared to those of the commercial product and once daily formulation (see FIG. 9).

The PK simulation results indicate that the dual release gastric retentive formulation can allow a once or twice daily administration which provides drug exposure similar to that provided by the commercial immediate release product administered 3 times daily. Twice daily administration was surprisingly effective in providing a less variable plasma profile as compared to the commercial immediate release product. The less variable plasma profile may be beneficial in minimizing adverse effects that may be associated with varied plasma levels of the active agent.

### Example 6

### Stability Testing of Levosulpiride Formulations

In the present work, levosulpiride was subjected to stress and compatibility studies using methods published in the European Pharmacopoeia, Supplement 2001, 1572 Assay. Both levosulpiride powder and granules comprising 95 wt % levosulpiride and 5 wt % povidone (PVP) prepared as described in Example 1 were incubated in open dishes in a stability chamber/oven at 50° C. or 80° C., 75% relative humidity (RH) for 3 days. The substances were then analyzed by HPLC (high performance liquid chromatography). The mobile phase (MP) was composed of 10% acetonitrile (ACN), 10% methanol (MeOH) and an 80% solution containing 6.8 g/l of potassium dihydrogen phosphate and 1 g/l of sodium octanesulphonate, pH 3.3. The column was a 0.25 m×4.6 mm, 5 μm Zorbax RX C8 (Agilent, Inc.), flow was set at 1.5 ml/min with a wavelength of 240 nm.

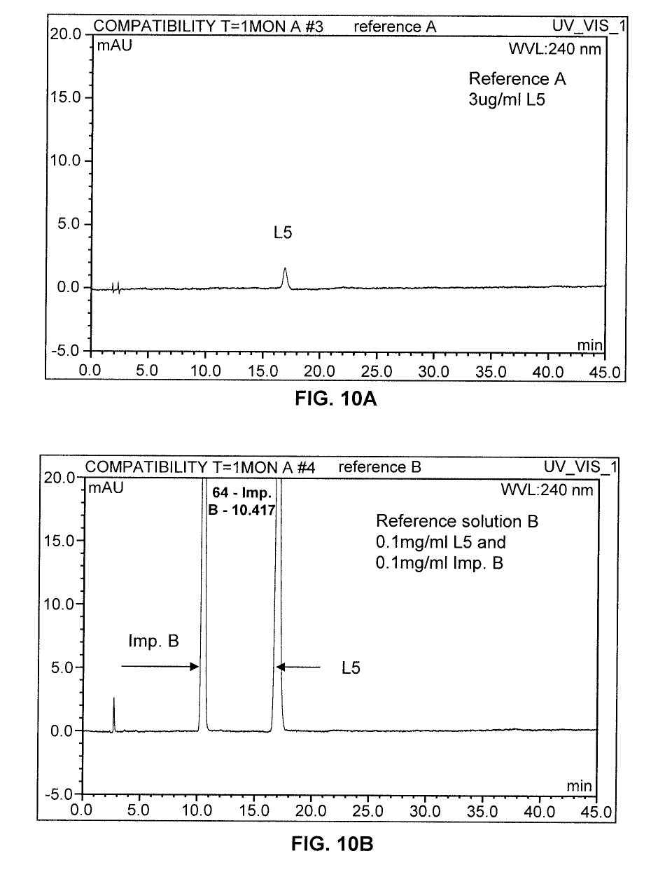

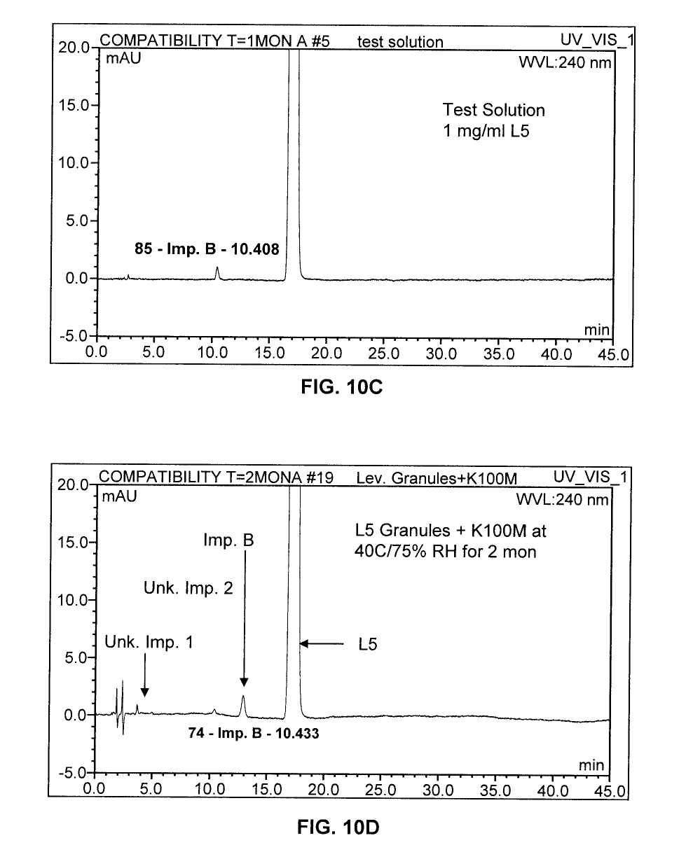

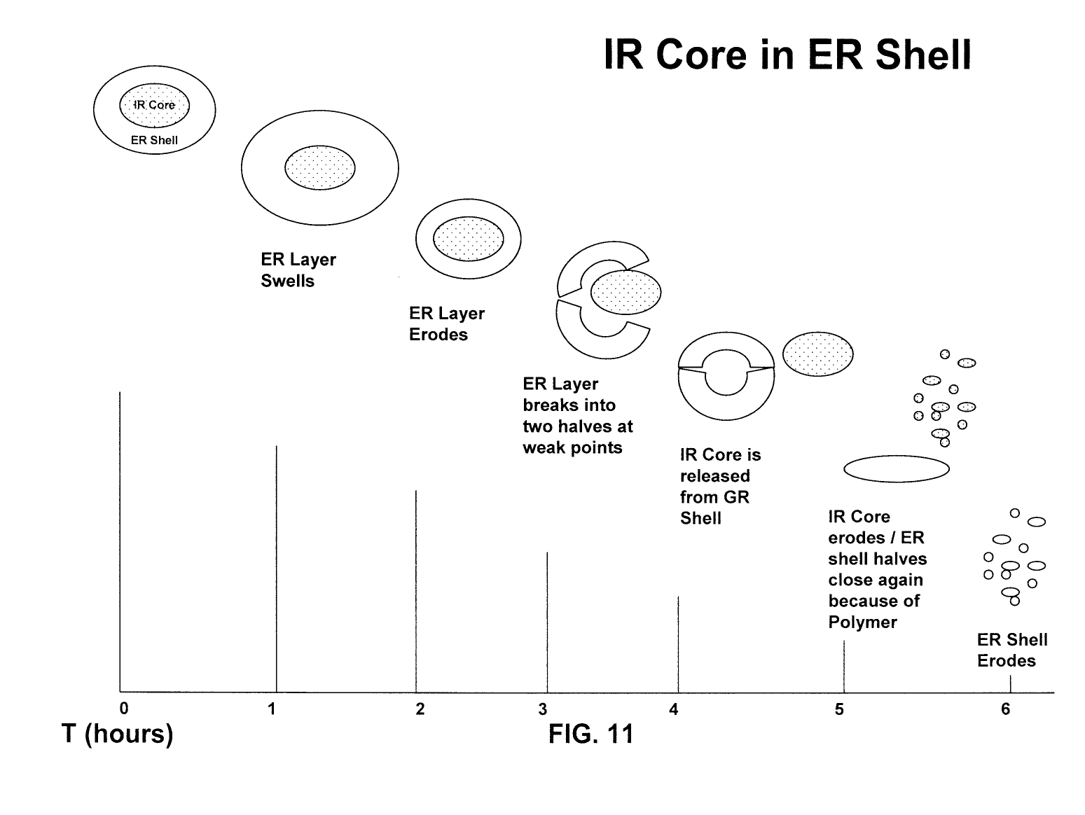

As shown in Tables 10 and 11, below, and FIGS. 10A-D, the levosulpiride impurities did not increase under the stressed condition for the duration of the study period. Furthermore, the granules with 5% povidone had the same level of impurities as the levosulpiride and the impurities of the granules did not increase under the stressed condition for the length of the study.

TABLE 10

| | | | | 50° C./75% | 80° C./75% |
|---|---|---|---|---|---|
| | | | Time = | RH, Time = | RH, Time = |
| | RT | | 0 | 3 days | 3 days |
| | (min) | RRT | RPA (%) | RPA (%) | RPA (%) |
| Levosulpiride | 17.08 | 1.00 | 100 | 100 | 100 |
| Impurity B | 10.15 | 0.59 | <0.05 | <0.05 | <0.05 |
| Unknown | 3.66 | 0.21 | <0.05 | <0.05 | <0.05 |
| Impurity 1 | | | | | |
| Unknown | 12.76 | 0.75 | 0.17 | 0.20 | 0.22 |
| Impurity 2 | | | | | |

TABLE 11

| | | Granules | | |
|---|---|---|---|---|
| | | | 50° C./75% | 80° C./75% |
| | Levosulpiride | | RH, Time = | RH, Time = |
| | Time = 0 | Time = 0 | 3 days | 3 days |
| | RPA (%) | RPA (%) | RPA (%) | RPA (%) |
| Levosulpiride | 100 | 100 | 100 | 100 |
| Impurity B | <0.05 | <0.05 | <0.05 | <0.05 |
| Unknown | <0.05 | <0.05 | <0.05 | <0.05 |
| Impurity 1 | | | | |
| Unknown | 0.17 | 0.19 | 0.22 | 0.21 |
| Impurity 2 | | | | |

### Example 7

### Levosulpiride Compatibility Study

Studies were done to determine compatibility of the levosulpiride with the various excipients used in the formulations described herein. Samples, as described in Table 12 below, were packaged into 60 cc high-density polyethylene (HDPE) bottles with an induction seal, then stored at 40° C., 75% relative humidity (RH). Samples may be taken at various time points to analyze for the presence of impurities.

TABLE 12
Description of Samples Prepared for Compatibility Determinations

| | |
|---|---|
| Sample Description | Notes |

Dry Blend

| | |
|---|---|
| Levosulpiride | Active ingredient |
| Levosulpiride + Povidone (PVP) | 1:1 |
| Levosulpiride + microcrystalline (MCC) + | 1:2:1 |
| Povidone | |

Active Granules + Excipients

| | |
|---|---|
| Levosulpiride Granules | Levosulpiride + 5% |
| | povidone granulated |
| Levosulpiride/MCC granules | Levosulpiride + 5% PVP + |
| | 63% MCC granulated |
| Levosulpiride granules + Polyox 1105 | 1:10 |
| Levosulpiride granules + Polyox N60k | 1:10 |
| Levosulpiride granules + Methocel K100M | 1:10 |
| Levosulpiride granules + Methocel K4M | 1:10 |
| Levosulpiride granules + MCC PH-101 | 1:5 |
| Levosulpiride granules + Mannitol | 1:5 |
| (Pearlitol DS 200) | |
| Levosulpiride granules + Crospovidone XL | 1:1 |

Various samples comprised of the levosulpiride with excipients as described in Table 11 below were packaged in 60 cc HDPE bottles with induction seals, then stored at 40° C., 75% RH. Samples were analyzed at 1 and 2 months using HPLC according to the method described in Example 6. Results are presented in Table 13 below and show that total impurities of the levosulpiride blends with binder and filler were 0.14-0.21%, which is comparable to the level for active alone.

TABLE 13

| | | Levosulpiride + | Levosulpiride + | Levosulpiride + |
|---|---|---|---|---|
| | Levosulpiride | PVP | MCC (1:2) | MCC + PVP |
| | 1 vs. 2 month | 1 vs. 2 month | 1 vs. 2 month | 1 vs. 2 month |
| | RPA (%) | RPA (%) | RPA (%) | RPA (%) |
| Levosulpiride | 100 | 100 | 100 | 100 |
| Impurity B | <0.05 | <0.05 | <0.05 | <0.05 |
| Unknown | <0.05 | <0.05 | <0.05 | <0.05 |
| Impurity 1 | | | | |
| Unknown | 0.18 vs. 0.19 | 0.14 v. 0.19 | 0.19 vs. 0.18 | 0.19 vs. 0.17 |
| Impurity 2 | | | | |

Compatibility Studies of levosulpiride with other excipients are shown in Tables 14 and 15 below. Total impurities of the granules physical blends with MCC, Mannitol, Crospovidone, Polyox 1105, Polyox N60k, Methocel K4M and Methocel K100M were 0.14-0.21% at 40° C., 75% RH for 1 and 2 months, which is comparable to the impurity levels for granules alone.

TABLE 14

| | Levosulpiride | Levosulpiride + | Levosulpiride + | Levosulpiride + |
|---|---|---|---|---|
| | granules alone | MCC | Mannitol | Crospovidone |
| | 1 vs. 2 month | 1 vs. 2 month | 1 vs. 2 month | 1 vs. 2 month |
| | RPA (%) | RPA (%) | RPA (%) | RPA (%) |
| Levosulpiride | 100 | 100 | 100 | 100 |
| Impurity B | <0.05 | <0.05 | <0.05 | <0.05 |
| Unknown | <0.05 | <0.05 | <0.05 | <0.05 |
| Impurity 1 | | | | |
| Unknown | 0.21 vs. 0.19 | 0.19 v. 0.19 | 0.20 vs. 0.14 | 0.19 vs. 0.21 |
| Impurity 2 | | | | |

TABLE 15
Compatibility Study

| | Levosulpiride + | Levosulpiride + | Levosulpiride + | Levosulpiride + |
|---|---|---|---|---|
| | Polyox 1105 | Polyox N60k | Methocel K4M | Methocel K100M |
| | 1 vs. 2 month | 1 vs. 2 month | 1 vs. 2 month | 1 vs. 2 month |
| | RPA (%) | RPA (%) | RPA (%) | RPA (%) |
| Levosulpiride | 100 | 100 | 100 | 100 |
| Impurity B | <0.05 | <0.05 | <0.05 | <0.05 |
| Unknown | <0.05 | <0.05 | <0.05 | <0.05 |
| Impurity 1 | | | | |
| Unknown | 0.19 vs. 0.17 | 0.14 v. 0.14 | 0.15 vs. 0.20 | 0.20 vs. 0.19 |
| Impurity 2 | | | | |

### Example 8

### Oral Dosage Form Providing a Delayed Immediate Burst of Levosulpiride

A gastric retained oral dosage form was formulated in which an immediate release core containing levosulpiride was completely encased by a “shell” formulated with a swellable hydrophilic polymer. This tablet was designed to provide extended release of levosulpiride followed by a “burst” of levosulpiride. This burst of levosulpiride prevents the blood levels of levosulpiride from decreasing to a therapeutically non-effective level.

The IR core (inner IR layer), was formulated as shown in Table 16 below. Levosulpiride was granulated with MCC PH-101, Mannitol, Plasdone and Ac-Di-Sol to form the active granules, which were then blended with additional Ac-Di-Sol, blue dye and magnesium stearate.

TABLE 16

| | Ingredient | mg (Wt %) | |
|---|---|---|---|
| Components of the | Levosulpiride | 19.00 | (97) |
| initial granulation | MCC PH-101 | 62.50 | (40.45) |
| mixture | Mannitol (Pearlitol 200) | 62.50 | (40.45) |
| | Plasdone K29/32 | 4.50 | (2.91) |
| | AcDiSol | 1.5 | (0.97) |
| Components blended | AcDiSol | 1.5 | (0.97) |
| with the granules | Blue dye | 1.5 | (0.97) |
| | Magnesium Stearate | 1.5 | (0.97) |
| | Total | 154.50 | (100) |

An ER shell was formulated such that it would swell in the stomach, slowly release the levosulpiride over several hours, then break apart to release the IR core. Three formulations were prepared for the ER shell layer as shown in Table 17 in order to test the effects of different hydrophilic polymers on the drug release and burst characteristics of the ER shell. Specifically, two polyethylene oxide (PEO) types, PEO WSR N-1105 (average molecular weight of 900,000 Da) and PEO WSR N-60K (average molecular weight of 2,000,000 Da) were used. Additionally, the PEO WSR N-1105 was present at two different percentages: 20 wt % and 40 wt %.

TABLE 17

| | GR burst 1 | | GR burst 2 | | GR burst 3 | |
|---|---|---|---|---|---|---|
| Ingredient | mg (wt %) | | mg (wt %) | | mg (wt %) | |
| Levosulpiride | 19.47 | (2.78) | 19.47 | (2.78) | 19.47 | (2.78) |
| granules | | | | | | |
| PEO WSR N-1105 | 140.00 | (20) | 280.00 | (40) | | |
| PEO WSR N-60K | — | | — | | 280.00 | (40) |
| MCC PH101 | 533.53 | (76.22) | 393.53 | (56.22) | 393.53 | (56.22) |
| Magnesium Stearate | 7.00 | (1) | 7.00 | (1) | 7.00 | (1) |
| Total | 700 | (100) | 700 | (100) | 700 | (100) |

To prepare each of the three tablet formulations, a mixture was first prepared using levosulpiride granules prepared according to the method described in Example 1. The granules were then blended with the AcDiSol, blue die and magnesium stearate, compressed using a Carver press at 1000 lb, 100% pump speed and 0 sec dwell time. The tooling used for compression is 0.3236″ round standard concave shape. Ac-Di-Sol was added in both intra and extra granules as superdisintegrant.

To prepare the extended release shell, the levosulpiride granules were prepared according to the method described in Example 1, then blended with the remaining excipients as described in Example 1. The tablets were then compressed by a manual hand crank mode on a Carver Press, tooled with a 0.4803″ round standard concave tooling such that the PEO-containing mixture completely encased the IR core. The tablets were compressed at 1000 lbs, 100% pump speed and 0 sec dwell time.

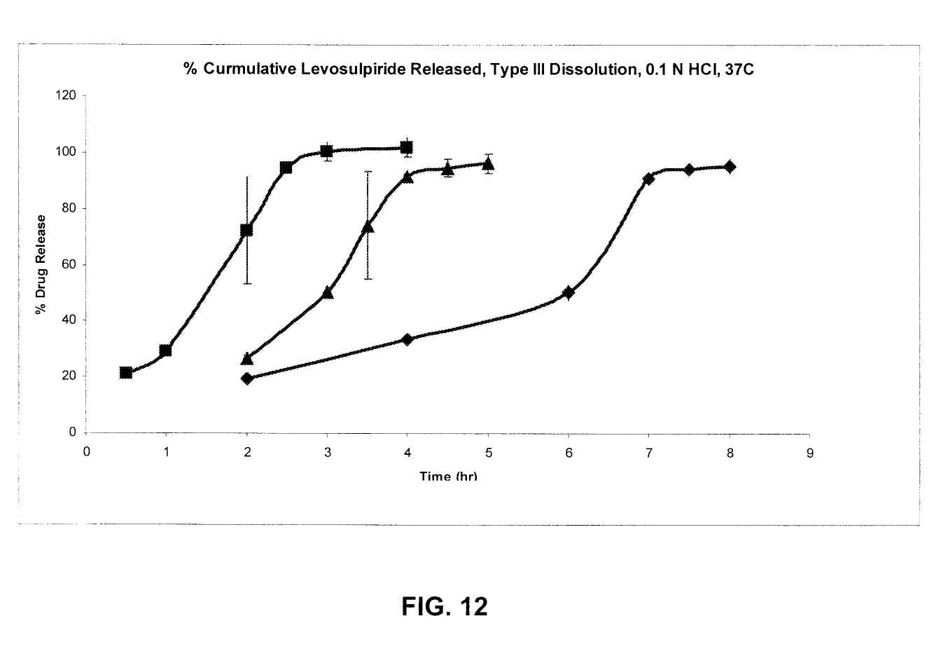

The levosulpiride release profiles of the resultant tablets were determined in an Apparatus III (250 ml) USP Disintegration Tester in 0.1 N HCl, 30 dpm (dips per minute) at 37±2° C. Results are presented below in FIG. 12 and clearly show that by using the PEO with the higher molecular weight provided a slower release of levosulpiride from the ER shell (compare tablets having 40 wt % PEO WSR N-1105 (▴) and those having 40 wt % PEO WSR N-60K (♦)). Additionally, an increase in the amount of high molecular weight hydrophilic polymer in the tablet slowed the release of levosulpiride from the ER shell (compare tablets having 20 wt % PEO WSR N-1105 (▪) and those having 40 wt % PEO WSR N-1105 (♦)). It is also important to note that the tablets having the PEO WSR N-60K polymer provided a greater time delay until burst of the levosulpiride from the IR core. Visual observations of the tablets having PEO N-60K and PEO N-1105 confirmed that the ER shell did swell and burst to release the IR core (data not shown).

Tablets were also formulated in which the IR core contained 6.5 mg levosulpiride and the ER shell contained 31 mg levosulpiride. The burst of the levosulpiride from the IR core was much less pronounced in these formulations, possibly due to the decreased amount of levosulpiride in the core.

All patents and patent publications referred to herein are hereby incorporated by reference.

Certain modifications and improvements will occur to those skilled in the art upon a reading of the foregoing description. It should be understood that all such modifications and improvements have been deleted herein for the sake of conciseness and readability but are properly within the scope of the following claims.

1. A gastric retentive dosage form comprising
an extended release (ER) layer comprising a first dose of levosulpiride dispersed in a polymeric matrix wherein the polymeric matrix comprises one or more polymers that upon imbibition of fluid swells to a size sufficient for gastric retention.

2. The dosage form of claim 1, further comprising an immediate release (IR) layer which comprises a second dose of levosulpiride.

3. The dosage form of claim 2, wherein the IR layer is a coating which is in contact with the entire surface of the ER layer.

4. The dosage form of claim 2, wherein the dosage form is a multilayer tablet, and wherein the IR layer is a second layer in the tablet.

5. The dosage form of claim 1, wherein the first dose of levosulpiride is about 50 mg to 100 mg.

6. The dosage form of claim 1, wherein the first dose of levosulpiride is released from the ER layer over a time period of about 6 to 12 hours.

7. The dosage form of claim 1, wherein the first dose of levosulpiride is released from the ER layer over a time period of about 6, about 8, or about 12 hours.

8. The dosage form of claim 1, wherein the first dose of levosulpiride is about 20 mg to 50 mg.

9. The dosage form of claim 8, wherein the first dose of levosulpiride is released from the ER layer over a time period of about 6 to about 8 hours.

10. The dosage form of claim 8, wherein the first dose of levosulpiride is released from the ER layer over a time period of about 6 or about 8 hours.

11. The gastric retentive dosage form of claim 1, wherein the ER layer comprises a polymeric matrix comprised of a poly(ethylene oxide) having an average molecular weight of about 900,000 Da to 10,000,000 Da.

12. The gastric retentive dosage form of claim 11, wherein the poly(ethylene oxide) is present in the ER layer at an amount of about 80 wt % of the ER layer.

13. A gastric retentive dosage form, comprising
an IR core, wherein said IR core contains a third dose of levosulpiride;
and an ER layer,
wherein the ER layer encases the IR core,
wherein the ER layer comprises a second dose of levosulpiride dispersed in a hydrophilic polymeric matrix,
wherein the polymeric matrix swells upon imbibition of fluid to a size at least 40% greater than the size of the polymeric matrix prior to oral administration,
and wherein the second dose of levosulpiride is released from the ER layer over a time period of about 4 to 8 hours.

14. The dosage form of claim 13, further comprising an IR coat.

15. The dosage form of claim 13, wherein the at least 90% of the levosulpiride is released from the IR core over a time period of about 30 to 60 minutes, and wherein the release of the levosulpiride from the IR coat begins about 6 hours after administration of the dosage form.

16. A method for treating a subject diagnosed with or suffering from dyspepsia or gastroesophageal reflux disease, with a dosage form comprising
an extended release (ER) layer comprising a second dose of levosulpiride dispersed in a polymeric matrix wherein the polymeric matrix comprises one or more polymers that upon imbibition of fluid swells to a size sufficient for gastric retention,
wherein the subject is in a fed mode.

17. The method of claim 16, wherein the dosage form further comprises an IR layer which comprises a first dose of levosulpiride

18. The method of claim 13, wherein the administering is once per 24 hour period.

19. The method of claim 13, wherein the administering is twice per 24 hour period.
